(19) United States
(12) Patent Application Publication
(10) Pub. No.: US 2009/0181921 A1
(43) Pub. Date: Jul. 16, 2009

(54) 2-5A ANALOGS AND THEIR METHODS OF USE

(75) Inventors: Lawrence Blatt, South San Francisco, CA (US); Leonid Beigelman, San Mateo, CA (US); Harri Lonnberg, Turku (FI)

Correspondence Address: KNOBBE MARTENS OLSON & BEAR LLP, 2040 MAIN STREET, FOURTEENTH FLOOR, IRVINE, CA, 92614, US

(73) Assignee: Alios BioPharma Inc., South San Francisco, CA (US)

(21) Appl. No.: 12/340,507

(22) Filed: Dec. 19, 2008

Related U.S. Application Data
(60) Provisional application No. 61/016,378, filed on Dec. 21, 2007.
(60) Provisional application No. 61/024,866, filed on Jan. 30, 2008.

(51) Int. Cl. A61K 31/7064; C07H 19/20; A61P 31/12

(52) U.S. Cl. 514/47; 536/26.7

(57) ABSTRACT

Disclosed herein are compounds that activate RNaseL, methods of synthesizing compounds that activate RNaseL and the use of compounds that activate RNaseL for treating and/or ameliorating a disease or a condition, such as a viral infection, cancer and/or parasitic disease.

This application claims the benefit of U.S. Provisional Application Nos. 61/016,378, entitled “2-5A ANALOGS AND THEIR METHODS OF USE” filed Dec. 21, 2007 and 61/024,866, entitled “2-5A ANALOGS AND THEIR METHODS OF USE” filed Jan. 30, 2008, both of which are incorporated by reference in their entireties, including any drawings.

### BACKGROUND

1. Field

This application relates to the fields of organic chemistry, pharmaceutical chemistry, biochemistry, molecular biology and medicine. In particular, disclosed herein are compounds that activate RNaseL, methods of synthesizing compounds that activate RNaseL, and the use of those compounds for treating and/or ameliorating a disease or a condition, such as a viral infection, parasitic infection and/or neoplastic disease.

2. Description of the Related Art

The interferon pathway is induced in mammalian cells in response to various stimuli, including viral infection. It is believed that this pathway induces the transcription of at least 200 molecules and cytokines, (immuno-regulatory substances that are secreted by cells of the immune system) involved in the defense against viral infections. These molecules and cytokines play a role in the control of cell proliferation, cell differentiation, and modulation of the immune responses.

The 2-5A system is one of the major pathways induced by the interferon pathway and has been implicated in some of its antiviral activities. This system has been described as comprising three enzymatic activities, including 2-5A-synthetases, 2-5A-phosphodiesterase, and RNaseL. 2-5A-synthetases are a family of four interferon-inducible enzymes which, upon activation by double-stranded RNA, convert ATP into the unusual series of oligomers known as 2-5A. The 2-5A-phosphodiesterase is believed to be involved in the catabolism of 2-5A from the longer oligomer. The 2-5A-dependent endoribonuclease L or RNase L is the effector enzyme of this system. RNaseL is normally inactive within the cell, so that it cannot damage the large amount of native RNA essential for normal cell function. Its activation by subnanomolar levels of 2-5A leads to the destruction of viral mRNA within the cell, and at the same time triggers the removal of the infected cell by inducing apoptosis (programmed cell death).

### SUMMARY

Some embodiments disclosed herein relate to a compound of Formula (I) or a pharmaceutically acceptable salt, prodrug or prodrug ester thereof:

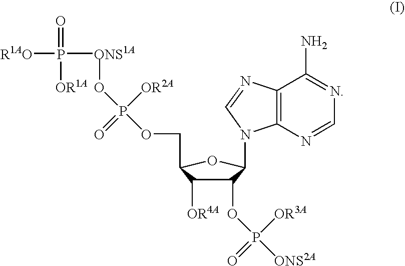

Other embodiments disclosed herein relate to a compound of Formula (Ia) or a pharmaceutically acceptable salt, prodrug or prodrug ester thereof:

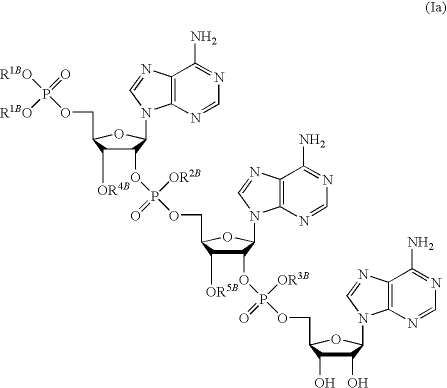

Some embodiments disclosed herein relate to methods of synthesizing a compound of Formula (I). Other embodiments disclosed herein relate to methods of synthesizing a compound of Formula (Ia).

Some embodiments disclosed herein relate to pharmaceutical compositions that can include one or more compounds of Formulae (I) and/or (Ia), and a pharmaceutically acceptable carrier, diluent, excipient or combination thereof.

Some embodiments disclosed herein relate to methods of ameliorating or treating a neoplastic disease that can include administering to a subject suffering from a neoplastic disease a therapeutically effective amount of one or more compound of Formulae (I) and/or (Ia) or a pharmaceutical composition that includes one or more compounds of Formulae (I) and/or (Ia).

Other embodiments disclosed herein relate to methods of inhibiting the growth of a tumor that can include administering to a subject having a tumor a therapeutically effective amount of one or more compound of Formulae (I) and/or (Ia) or a pharmaceutical composition that includes one or more compounds of Formulae (I) and/or (Ia).

Still other embodiments disclosed herein relate to methods of ameliorating or treating a viral infection that can include administering to a subject suffering from a viral infection a therapeutically effective amount of one or more compound of Formulae (I) and/or (Ia) or a pharmaceutical composition that includes one or more compounds of Formulae (I) and/or (Ia).

Yet still other embodiments disclosed herein relate to methods of ameliorating or treating a parasitic disease that can include administering to a subject suffering from a parasitic disease a therapeutically effective amount of one or more compound of Formulae (I) and/or (Ia) or a pharmaceutical composition that includes one or more compounds of Formulae (I) and/or (Ia).

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R¹, R^{1a} and R^{1b}, represent substituents that can be attached to the indicated atom. A non-limiting list of R groups include, but are not limited to, hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. An R group may be substituted or unsubstituted. If two “R” groups are covalently bonded to the same atom or to adjacent atoms, then they may be “taken together” as defined herein to form a cycloalkyl, cycloalkenyl, cycloalkynyl, aryl, heteroaryl or heteroalicyclyl group. For example, without limitation, if Rₐ and R_{b} of an NRₐR_{b} group are indicated to be “taken together”, it means that they are covalently bonded to one another at their terminal atoms to form a ring that includes the nitrogen:

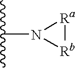

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents.

The term “substituted” has its ordinary meaning, as found in numerous contemporary patents from the related art. See, for example, U.S. Pat. Nos. 6,509,331; 6,506,787; 6,500,825; 5,922,683; 5,886,210; 5,874,443; and 6,350,759; all of which are incorporated herein by reference for the limited purpose of disclosing suitable substituents that can be on a substituted group and standard definitions for the term “substituted.” Examples of suitable substituents include but are not limited to hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Each of these substituents can be further substituted. The other above-listed patents also provide standard definitions for the term “substituted” that are well-understood by those of skill in the art.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution unless otherwise indicated.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution unless otherwise indicated.

As used herein, “aryl” refers to a carbocyclic (all carbon) monocyclic or multicyclic aromatic ring system that has a fully delocalized pi-electron system. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof, unless the substituent groups are otherwise indicated.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

An “aralkyl” is an aryl group connected, as a substituent, via a lower alkylene group. The lower alkylene and aryl group of an aralkyl may be substituted or unsubstituted. Examples include but are not limited to benzyl, substituted benzyl, 2-phenylalkyl, 3-phenylalkyl, and naphtylalkyl.

A “heteroaralkyl” is heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their substituted as well as benzo-fused analogs.

“Lower alkylene groups” are straight-chained tethering groups, forming bonds to connect molecular fragments via their terminal carbon atoms. Examples include but are not limited to methylene (—CH₂—), ethylene (—CH₂CH₂—), propylene (—CH₂CH₂CH₂—), and

As used herein, “cycloalkyl” refers to a completely saturated (no double or triple bonds) mono- or multi-cyclic hydrocarbon ring system. When composed of two or more rings, the rings may be joined together in a fused, bridged or spiro-connected fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be selected from those substituents indicated above with respect to substitution of an aryl group unless otherwise indicated.

As used herein, “cycloalkenyl” refers to a cycloalkyl group that contains one or more double bonds in the ring; although, if there is more than one, the double bonds cannot form a fully delocalized pi-electron system (otherwise the group would be “aryl,” as defined herein). When composed of two or more rings, the rings may be connected together in a fused, bridged or spiro-connected fashion. A cycloalkenyl group may be unsubstituted or substituted. When substituted, the substituent(s) may be selected from the substituents disclosed above with respect to an aryl group substitution unless otherwise indicated.

As used herein, “cycloalkynyl” refers to a cycloalkyl group that contains one or more triple bonds in the ring. If there is more than one triple bond, the triple bonds cannot form a fully delocalized pi-electron system. When composed of two or more rings, the rings may be joined together in a fused, bridged or spiro-connected fashion. A cycloalkynyl group may be unsubstituted or substituted. When substituted, the substituent(s) may be selected from the substituents disclosed above with respect to an aryl group substitution unless otherwise indicated.

As used herein, “heteroalicyclic” or “heteroalicyclyl” refers to a stable 3- to 18 membered monocyclic, bicyclic, tricyclic, or tetracyclic ring system which consists of carbon atoms and from one to five heteroatoms such as nitrogen, oxygen and sulfur. The “heteroalicyclic” or “heteroalicyclyl” may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl or heteroalicyclic groups may be unsubstituted or substituted. When substituted, the substituent(s) may be one or more groups independently selected from: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” groups include but are not limited to, azepinyl, acridinyl, carbazolyl, cinnolinyl, 1,3-dioxin, 1,3-dioxane, 1,4-dioxane, 1,2-dioxolanyl, 1,3-dioxolanyl, 1,4-dioxolanyl, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazolinyl, imidazolidine, isoxazoline, isoxazolidine, oxazoline, oxazolidine, oxazolidinone, thiazoline, thiazolidine, morpholinyl, oxiranyl, piperidinyl N-Oxide, piperidinyl, piperazinyl, pyrrolidinyl, pyrrolidone, pyrrolidione, 4-piperidonyl, pyrazoline, pyrazolidinyl, 2-oxopyrrolidinyl, tetrahydropyran, 4H-pyran, tetrahydrothiopyran, thiamorpholinyl, thiamorpholinyl sulfoxide, thiamorpholinyl sulfone, and their benzo-fused analogs (e.g., benzimidazolidinone, tetrahydroquinoline, 3,4-methylenedioxyphenyl).

A “(heteroalicyclyl)alkyl” is a heterocyclic or a heteroalicyclylic group connected, as a substituent, via a lower alkylene group. The lower alkylene and heterocyclic or a heterocyclyl of a (heteroalicyclyl)alkyl may be substituted or unsubstituted. Examples include but are not limited tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl.

As used herein, “alkoxy” refers to the formula —OR wherein R is an alkyl is defined as above, e.g. methoxy, ethoxy, n-propoxy, 1-methylethoxy (isopropoxy), n-butoxy, iso-butoxy, sec-butoxy, tert-butoxy, and the like. An alkoxy may be substituted or unsubstituted.

As used herein, “acyl” refers to a hydrogen, alkyl, alkenyl, alkynyl, or aryl connected, as substituents, via a carbonyl group. Examples include formyl, acetyl, propanoyl, benzoyl, and acryl. An acyl may be substituted or unsubstituted.

As used herein, “hydroxyalkyl” refers to an alkyl group in which one or more of the hydrogen atoms are replaced by hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by halogen (e.g., mono-haloalkyl, di-haloalkyl and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “aryloxy” and “arylthio” refers to RO— and RS—, in which R is an aryl, such as but not limited to phenyl. Both an aryloxy and arylthio may be substituted or unsubstituted.

A “sulfenyl” group refers to an “—SR” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein X is a halogen.

A “trihalomethanesulfonamido” group refers to an “X₃CS(O)₂ R_{A}N—” group wherein X is a halogen and R_{A} defined with respect to O-carboxy.

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂NR_{A}R_{B}” group in which R_{A} and R_{B} can be the same as R defined with respect to O-carboxy. An S-sulfonamido may be substituted or unsubstituted.

An “N-sulfonamido” group refers to a “RSO₂N(R_{A})—” group in which R and R_{A} can be the same as R defined with respect to O-carboxy. A N-sulfonamido may be substituted or unsubstituted.

An “O-carbamyl” group refers to a “—OC(═O)NR_{A}R_{B}” group in which R_{A} and R_{B} can be the same as R defined with respect to O-carboxy. An O-carbamyl may be substituted or unsubstituted.

An “N-carbamyl” group refers to an “ROC(═O)NR_{A}—” group in which R and R_{A} can be the same as R defined with respect to O-carboxy. An N-carbamyl may be substituted or unsubstituted.

An “O-thiocarbamyl” group refers to a “—OC(═S)—NR_{A}R_{B}” group in which R_{A} and R_{B} can be the same as R defined with respect to O-carboxy. An O-thiocarbamyl may be substituted or unsubstituted.

An “N-thiocarbamyl” group refers to an “ROC(═S)NR_{A}—” group in which R and R_{A} can be the same as R defined with respect to O-carboxy. An N-thiocarbamyl may be substituted or unsubstituted.

A “C-amido” group refers to a “—C(═O)NR_{A}R_{B}” group in which R_{A} and R_{B} can be the same as R defined with respect to O-carboxy. A C-amido may be substituted or unsubstituted.

An “N-amido” group refers to a “ROC(═O)NR_{A}—” group in which R and R_{A} can be the same as R defined with respect to O-carboxy. An N-amido may be substituted or unsubstituted.

As used herein, the term “levulinoyl” refers to a —C(═O)CH₂CH₂C(═O)CH₃ group.

The term “halogen atom,” as used herein, means any one of the radio-stable atoms of column 7 of the Periodic Table of the Elements, i.e., fluorine, chlorine, bromine, or iodine, with bromine and chlorine being preferred.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

As used herein, the term “nucleoside” refers to a compound composed of any pentose or modified pentose moiety attached to a specific portion of a heterocyclic base, tautomer, or derivative thereof such as the 9-position of a purine, 1-position of a pyrimidine, or an equivalent position of a heterocyclic base derivative. Examples include, but are not limited to, a ribonucleoside comprising a ribose moiety and a deoxyribonucleoside comprising a deoxyribose moiety. In some instances, the nucleoside can be a nucleoside drug analog.

As used herein, the term “nucleoside drug analog” refers to a compound composed of a nucleoside that has therapeutic activity, such as antiviral, anti-neoplastic, anti-parasitic and/or antibacterial activity.

As used herein, the term “nucleotide” refers to a nucleoside having a phosphate ester substituted on the 5′-position or an equivalent position of a nucleoside derivative.

As used herein, the terms “protected nucleoside” and “protected nucleoside derivative” refers to a nucleoside and nucleoside derivative, respectively, in which one or more hydroxy groups attached to the ribose or deoxyribose ring are protected with one or more protecting groups. An example of protected nucleoside is an adenosine in which the oxygen at the 3′-position is protected with a protecting group such as methyl group or a levulinoyl group.

As used herein, the term “heterocyclic base” refers to a purine, a pyrimidine and derivatives thereof. The term “purine” refers to a substituted purine, its tautomers and analogs thereof. Similarly, the term “pyrimidine” refers to a substituted pyrimidine, its tautomers and analogs thereof. Exemplary purines include, but are not limited to, purine, adenine, guanine, hypoxanthine, xanthine, theobromine, caffeine, uric acid and isoguanine. Examples of pyrimidines include, but are not limited to, cytosine, thymine, uracil, and derivatives thereof. An example of an analog of a purine is 1,2,4-triazole-3-carboxamide.

Other non-limiting examples of heterocyclic bases include diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-fluorouracil, 5-bromouracil, pseudoisocytosine, isocytosine, isoguanine, and other heterocyclic bases described in U.S. Pat. Nos. 5,432,272 and 7,125,855, which are incorporated herein by reference for the limited purpose of disclosing additional heterocyclic bases.

As used herein, the term “protected heterocyclic base” refers to a heterocyclic base in which one or more amino groups attached to the base are protected with one or more suitable protecting groups and/or one or more —NH groups present in a ring of the heterocyclic base are protected with one or more suitable protecting groups. When more than one protecting group is present, the protecting groups can be the same or different.

The terms “derivative,” “variant,” or other similar terms refer to a compound that is an analog of the other compound.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls (e.g., t-butoxycarbonyl (BOC)); arylalkylcarbonyls (e.g., benzyloxycarbonyl, benzoyl); substituted methyl ether (e.g. methoxymethyl ether); substituted ethyl ether; a substituted benzyl ether; tetrahydropyranyl ether; silyl ethers (e.g., trimethylsilyl, triethylsilyl, triisopropylsilyl, t-butyldimethylsilyl, or t-butyldiphenylsilyl); esters (e.g. benzoate ester); carbonates (e.g. methoxymethylcarbonate); sulfonates (e.g. tosylate, mesylate); acyclic ketal (e.g. dimethyl acetal); cyclic ketals (e.g., 1,3-dioxane or 1,3-dioxolanes); acyclic acetal; cyclic acetal; acyclic hemiacetal; cyclic hemiacetal; and cyclic dithioketals (e.g., 1,3-dithiane or 1,3-dithiolane).

“Leaving group” as used herein refers to any atom or moiety that is capable of being displaced by another atom or moiety in a chemical reaction. More specifically, in some embodiments, “leaving group” refers to the atom or moiety that is displaced in a nucleophilic substitution reaction. In some embodiments, “leaving groups” are any atoms or moieties that are conjugate bases of strong acids. Examples of suitable leaving groups include, but are not limited to, tosylates and halogens. Non-limiting characteristics and examples of leaving groups can be found, for example in Organic Chemistry, 2d ed., Francis Carey (1992), pages 328-331; Introduction to Organic Chemistry, 2d ed., Andrew Streitwieser and Clayton Heathcock (1981), pages 169-171; and Organic Chemistry, 5^{th} ed., John McMurry (2000), pages 398 and 408; all of which are incorporated herein by reference for the limited purpose of disclosing characteristics and examples of leaving groups.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference for the limited purpose describing procedures and preparation of suitable prodrug derivatives.

The term “pro-drug ester” refers to derivatives of the compounds disclosed herein formed by the addition of any of several ester-forming groups that are hydrolyzed under physiological conditions. Examples of pro-drug ester groups include pivaloyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference for the limited purpose of disclosing ester-forming groups that can form prodrug esters.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound described herein having one or more double bond(s) generating geometrical isomers that can be defined as E or Z, each double bond may independently be E or Z a mixture thereof. Likewise, all tautomeric forms are also intended to be included.

Some embodiments disclosed herein relates to a compound of Formula (I) as shown herein, or a pharmaceutically acceptable salt, prodrug or prodrug ester in which each R^{1A} can be

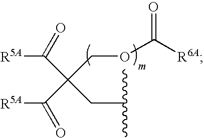

### R^{2A} can be R^{3A}

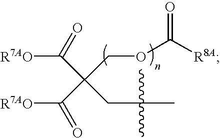

can be

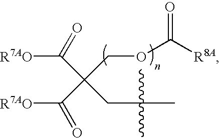

wherein R^{2A} and R^{3A} can be the same or different; R^{4A} can be —H or —C(R^{9A})₂—O—C(═O)R^{10A}; each R^{5A}, each R^{6A}, each R^{7A}, each R^{8A}, each R^{9A} and R^{10A} can be each independently hydrogen or an optionally substituted C₁₋₄-alkyl; each m can be independently 1 or 2; each n can be independently 1 or 2; NS^{1A} and NS^{2A} can be independently selected from a nucleoside, a protected nucleoside, a nucleoside derivative and a protected nucleoside derivative.

In an embodiment, each m can be 1. In another embodiment, each m can be 2. In some embodiments, each n can be 1. In other embodiments, each n can be 2. In an embodiment, each m and each n can be 1. In another embodiment, each m and each n can be 2. In some embodiments, m and n are not the same. In an embodiment, at least one m can be 1. In some embodiments, at least one n can be 1. In an embodiment, at least one m can be 2. In some embodiments, at least one n can be 2.

With respect to compounds of Formula (I), in some embodiments, each R^{5A} can be an optionally substituted C₁₋₄ alkyl. In an embodiment, both R^{5A} groups can be the same. In another embodiment, both R^{5A} groups can be the different. In some embodiments, R^{6A} can be an optionally substituted C₁₋₄ alkyl. In an embodiment, each R^{6A} can be methyl or tert-butyl. The R^{1A} groups of a compound of Formula (I) can be the same or different. Suitable R^{1A} groups include, but are not limited to, the following:

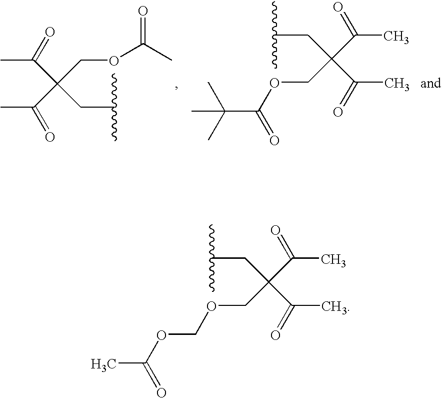

In some embodiments, each R^{7A} can be an optionally substituted C₁₋₄ alkyl. In an embodiment, both R^{7A} groups can be the same. In another embodiment, both R^{7A} groups can be the different. In some embodiments, each R^{8A} can be an optionally substituted C₁₋₄ alkyl. In an embodiment, R^{8A} can be methyl or tert-butyl.

Examples of suitable R^{2A} groups include, but are not limited to:

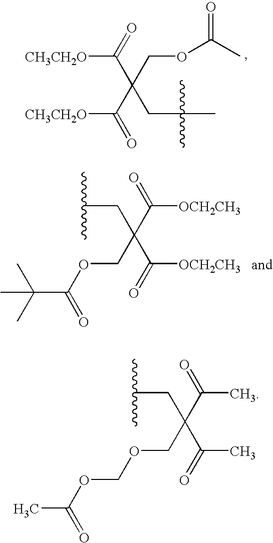

In some embodiments, the R^{3A} group can also be:

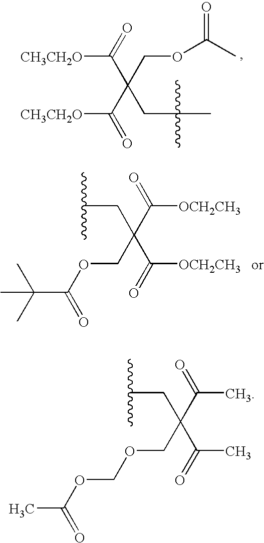

In some embodiments, R^{4A} can be —C(R^{9A})₂—O—C(═O)R^{10A} in which both R^{9A} groups can be hydrogen and R^{10A} can be an optionally substituted C₁₋₄ alkyl such as methyl or tert-butyl.

In some embodiments, NS^{1A} can be selected from anti-neoplastic agent, an anti-viral agent and an anti-parasitic agent. The anti-viral agent can be activity against various viruses, including, but not limited to, one or more of the following: an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and/or a Togaviridae. When NS^{1A} is an anti-neoplastic agent, in some embodiments, the compound of Formula (I) can have activity against cancer, tumors (e.g., solid tumors) and the like. Similarly, when NS^{1A} is an anti-parasitic agent, in an embodiment, the compound of Formula (I) can have activity against Chagas' disease.

An exemplary structure of NS^{1A} is:

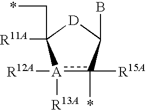

in which [symbol] can be a double or single bond; A can be selected from C (carbon), O (oxygen) and S (sulfur); B can be an optionally substituted heterocyclic base or a derivative thereof; D can be C═CH₂ or O (oxygen); R^{11A} can be selected from hydrogen, azido, —CN, an optionally substituted C₁₋₄ alkyl and an optionally substituted C₁₋₄ alkoxy; R^{12A} can be absent or selected from hydrogen, halogen, hydroxy and an optionally substituted C₁₋₄ alkyl; R^{13A} can be absent or selected from hydrogen, halogen, azido, amino, hydroxy, an optionally substituted C₁₋₄ alkoxy and —OC(R^{16A})₂—O—C(═O)R^{17A}; R^{15A} can be absent or selected from hydrogen, halogen, hydroxy, —CN, —NC, an optionally substituted C₁₋₄ alkyl, an optionally substituted haloalkyl and an optionally substituted hydroxyalkyl; each R^{16A} and R^{17A} can be independently hydrogen or an optionally substituted C₁₋₄-alkyl; and * represents a point of attachment.

The substituent R^{13A}, in some embodiments, can be an optionally substituted C₁₋₄ alkoxy. In an embodiment, R^{13A} can be —OCH₃. In other embodiments, R^{13A} can be —OC(R^{16A})₂—O—C(═O)R^{17A}. In an embodiment, when R^{13A} can be —OC(R^{16A})₂—O—C(═O)R^{1A}, both R^{16A} groups can be hydrogen and R^{17A} can be an optionally substituted alkyl (e.g., methyl).

In some embodiments, the heterocyclic base or derivative thereof represented by B can be selected from:

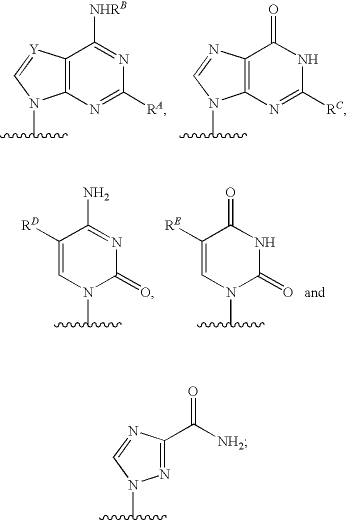

in which R^{A} can be hydrogen or halogen; R^{B} can be hydrogen, an optionally substituted C₁₋₄alkyl, or an optionally substituted C₃₋₈ cycloalkyl; R^{C} can be hydrogen or amino; R^{D} can be hydrogen or halogen; R^{E} can be hydrogen or an optionally substituted C₁₋₄ alkyl; and Y can be N (nitrogen) or CR^{F}, wherein R^{F} hydrogen, halogen or an optionally substituted C₁₋₄-alkyl.

Examples of suitable NS^{1A} groups include, but are not limited to, the following:

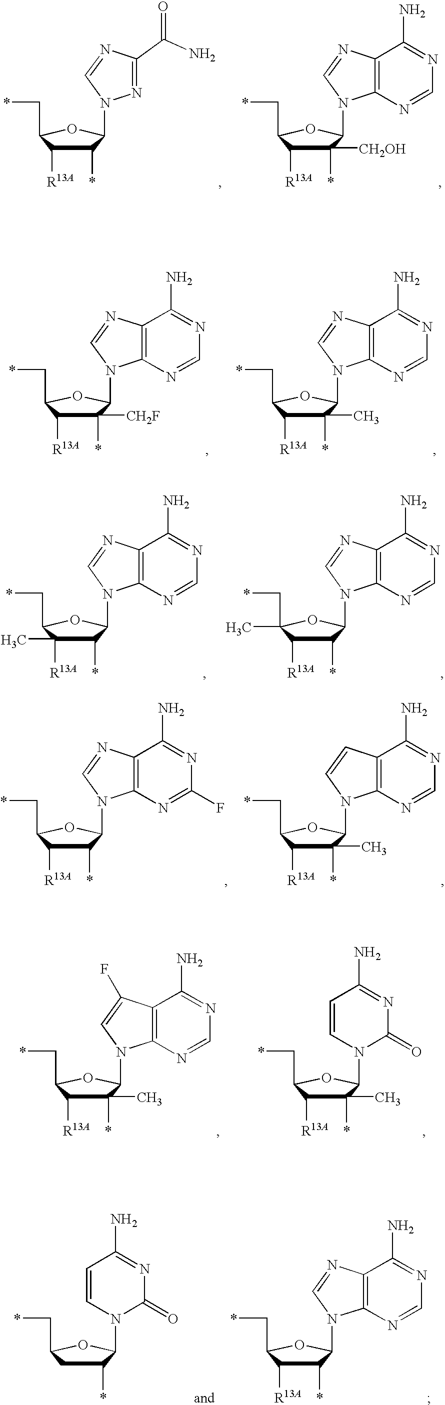

in which * represents a point of attachment; and R^{13A} can be absent or selected from hydrogen, halogen, azido, amino, hydroxy, an optionally substituted C₁₋₄ alkoxy and —OC(R^{16A})₂—O—C(═O)R^{17A}. In some embodiments, R^{13A} can be an optionally substituted C₁₋₄ alkoxy, for example, —OCH₃. In other embodiments, R^{13A} can be —OC(R^{16A})₂—OC(═O)R^{17A}. In an embodiment, when R^{13A} can be —OC(R^{16A})₂—O—C(═O)R^{17A}, both R^{16A} groups can be hydrogen and R^{17A} can be an optionally substituted C₁₋₄ alkyl (e.g., methyl).

Similar to NS^{1A}, in some embodiments, NS^{2A} can be selected from anti-neoplastic agent, an anti-viral agent and an anti-parasitic agent. An exemplary structure of NS^{2A} is:

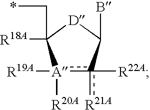

in which [symbol] can be a double or single bond; A″ can be selected from C (carbon), O (oxygen) and S (sulfur); B″ can be an optionally substituted heterocyclic base or a derivative thereof, D″ can be C═CH₂ or O (oxygen); R^{18A} can be selected from hydrogen, azido, —CN, an optionally substituted C₁₋₄ alkyl and an optionally substituted C₁₋₄ alkoxy; R^{19A} can be absent or selected from hydrogen, halogen, hydroxy and an optionally substituted C₁₋₄ alkyl; R^{20A} can be absent or selected from hydrogen, halogen, azido, amino and hydroxy; R^{21A} can be selected from hydrogen, halogen, hydroxy, —CN, —NC, an optionally substituted C₁₋₄ alkyl and an optionally substituted C₁₋₄ alkoxy; R^{22A} can be absent or selected from hydrogen, halogen, hydroxy, —CN, —NC, an optionally substituted C₁₋₄ alkyl, an optionally substituted haloalkyl and an optionally substituted hydroxyalkyl, or when the bond to R^{21A} indicated by [symbol] is a double bond, then R^{21A} is a C₁₋₄ alkenyl and R^{22A} is absent; and * represents a point of attachment.

In some embodiments, the optionally substituted heterocyclic base or a derivative thereof, B″, can be selected from one of the following:

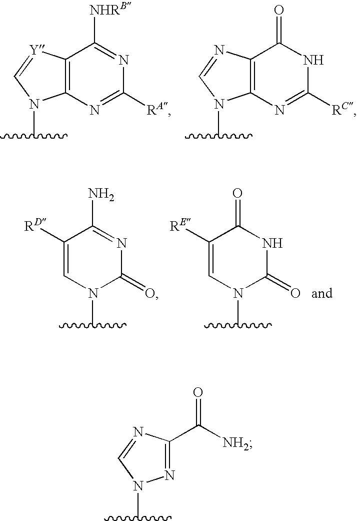

in which R^{A″} can be hydrogen or halogen; R^{B″} can be hydrogen, an optionally substituted C₁₋₄ alkyl, or an optionally substituted C₃₋₈ cycloalkyl; R^{C″} can be hydrogen or amino; R^{D″} can be hydrogen or halogen; R^{E″} can be hydrogen or an optionally substituted C₁₋₄alkyl; and Y can be N (nitrogen) or CR^{F″}, wherein R^{F″} hydrogen, halogen or an optionally substituted C₁₋₄-alkyl.

Suitable examples of NS^{2A} include, but are not limited to, the following:

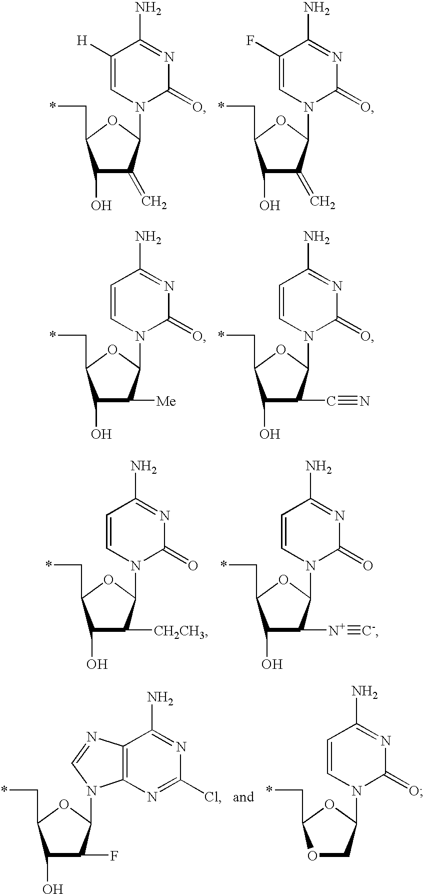

in which * represents a point of attachment.

Additional examples of NS^{2A} include the following:

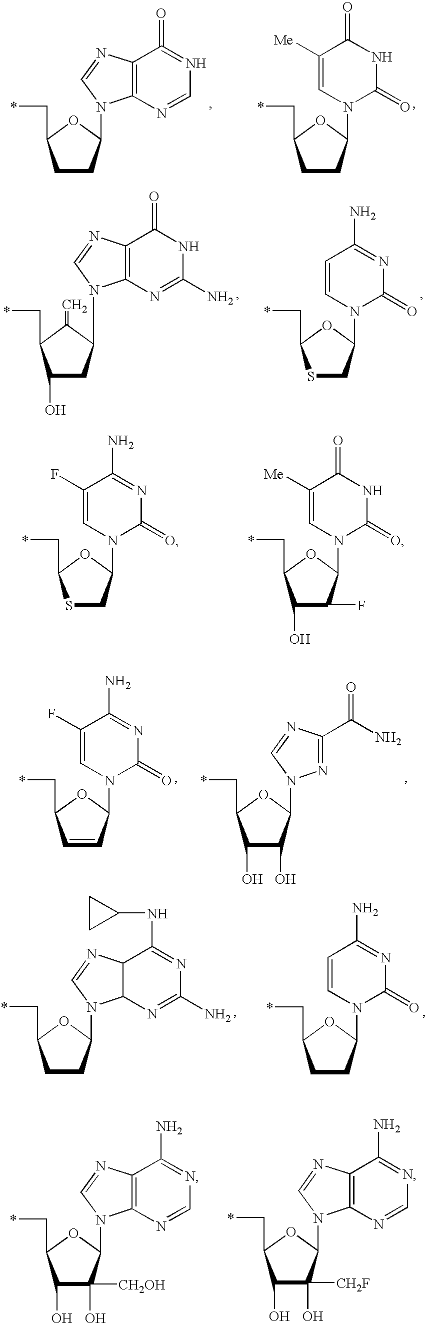

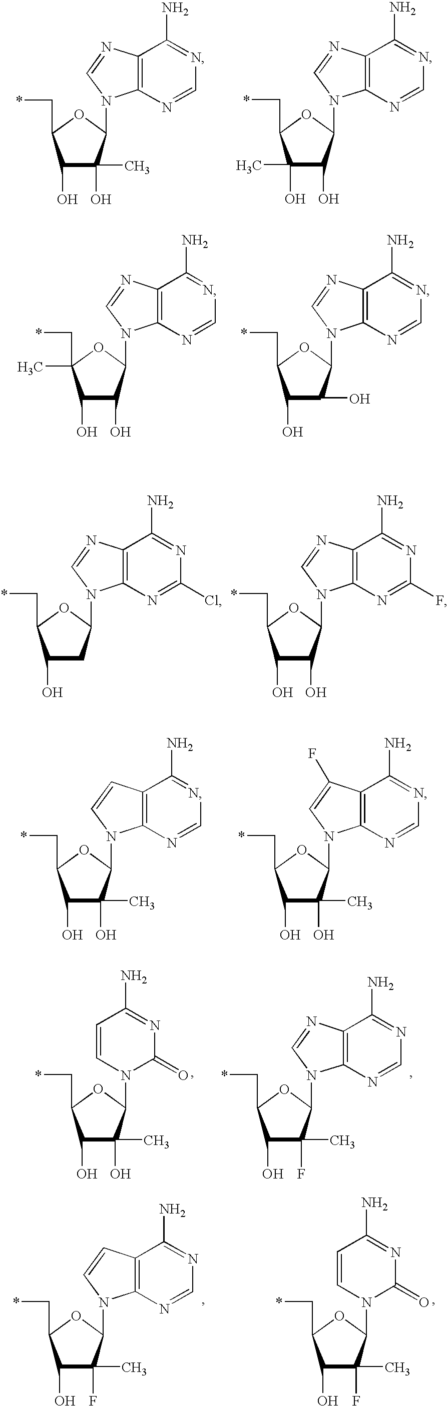

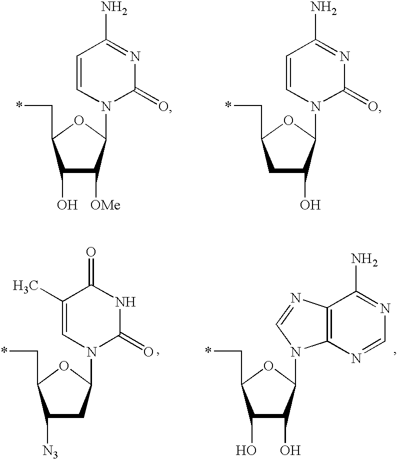

in which * represents a point of attachment.

In some embodiments, the compound of Formula (I) can have NS^{1A} as

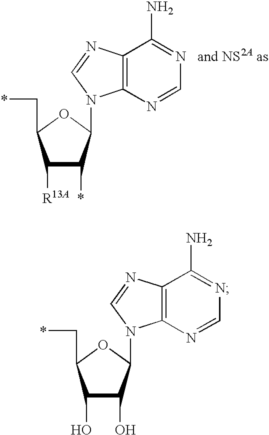

in which R^{13A} can be selected from —OH, an optionally substituted C₁₋₄ alkoxy and —OC(R^{16A})₂—O—C(═O)R^{17A}; each R^{16A} and R^{17A} can be independently hydrogen or an optionally substituted C₁₋₄-alkyl; and * represents a point of attachment. In an embodiment, R^{13A} can be —OC(R^{16A})₂—O—C(═O)R^{17A}. In some embodiments when R^{13A} is —OC(R^{16A})₂—O—C(═O)R^{17A}, then both R^{16A} groups can be hydrogen and R^{17A} can be an optionally substituted C₁₋₄-alkyl, such as methyl. In another embodiment, R^{13A} can be an optionally substituted C₁₋₄ alkoxy, such as methoxy.

As previously stated, NS^{1A} and/or NS^{2A} can be an anti-viral agent, an anti-neoplastic agent and/or an anti-parasitic agent. In an embodiment, the anti-viral agent, anti-neoplastic agent and anti-parasitic agent can be selected to target a particular virus, tumor or parasite, thereby providing a dual mode of action. Upon administration of one or more compounds of Formula (I) to an animal, such as a human, a non-human mammal, a bird, or another animal, the full molecule can activate RNaseL, producing a general anti-viral response, and upon degradation of the compound in vivo, the nucleoside(s) is released, thus generating the particular (generally more specific) therapeutic action (e.g., anti-viral, anti-neoplastic and/or anti-parasitic action) of that moiety. Further, upon release of the nucleoside(s), the intracellular cleavage releases not a nucleoside, but its active, phosphorylated form. This not only makes the nucleoside(s) more immediately available in the intracellular environment, but also bypasses some potential resistance mechanisms such as those described herein. One mechanism that is bypassed is the need for kinase-mediated phosphorylation that both reduces the efficacy of nucleosides in general, but also provides a potential resistance mechanism. This dual-mode of action can provide a powerful benefit in addressing difficult neoplasms, viral infections and/or parasitic infections.

Other embodiments disclosed herein relates to a compound of Formula (Ia) as shown herein, or a pharmaceutically acceptable salt, prodrug or prodrug ester in which each R^{1B} can be

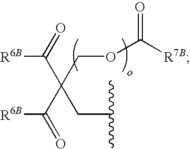

### R^{2B} can be

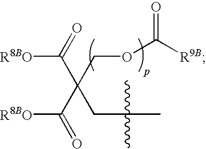

### R^{3B} can be

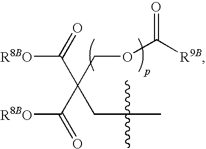

wherein R^{2B} and R^{3B} can be the same or different; R^{4B} and R^{5B} can be independently selected from hydrogen, an optionally substituted C₁₋₄ alkyl, and —C(R^{10B})₂—O—C(═O)R^{11B}; each R^{6B}, each R^{7B}, each R^{8B}, each R^{9B}, each R^{10B} and each R^{11B} can be each independently hydrogen or an optionally substituted C₁₋₄-alkyl; each o can be independently 1 or 2; and each p can be independently 1 or 2.

In an embodiment, each o can be 1. In another embodiment, each o can be 2. In some embodiments, each p can be 1. In other embodiments, each p can be 2. In an embodiment, each o and each p can be 1. In another embodiment, each o and each p can be 2. In some embodiments, o and p are different. In an embodiment, at least one o can be 1. In some embodiments, at least one p can be 1. In an embodiment, at least one o can be 2. In some embodiments, at least one p can be 2.

In some embodiments, each R^{6B} can be an optionally substituted C₁₋₄ alkyl. In an embodiment, both R^{6B} groups can be the same. In another embodiment, the R^{6B} groups can be different. In some embodiments, each R^{7B} can be an optionally substituted C₁₋₄ alkyl such as methyl or tert-butyl. Examples of R^{1B} include, but are not limited to, the following:

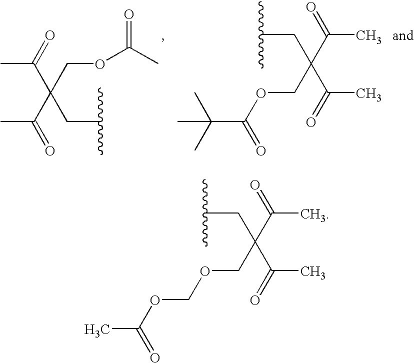

In some embodiments, each R^{8B} can be an optionally substituted C₁₋₄ alkyl. In an embodiment, both R^{8B} groups can be the same. In another embodiment, the R^{8B} groups can be different. In some embodiments, each R^{9B} can be an optionally substituted C₁₋₄ alkyl. In an embodiment, R^{9B} can be methyl or tert-butyl. Exemplary R^{2B} and R^{3B} groups include, but are not limited to the following:

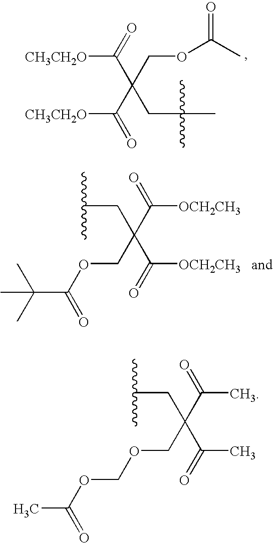

The 3′-position on the 5′-terminal residue, R^{4B}, in some embodiments of Formula (Ia), R^{4B} can be an optionally substituted C₁₋₄ alkyl. In an embodiment, R^{4B} can be methyl. In other embodiments, R^{4B} can be —C(R^{10B})₂—O—C(═O)R^{11B}. In an embodiment when R^{4B} is —C(R^{10B})₂—O—C(═O)R^{11B}, then both R^{10B} can be hydrogen and R^{11B} can be an optionally substituted C₁₋₄ alkyl, for example, methyl.

For the 3′-position on the middle residue, in some embodiments, R^{5B} can be —C(R^{10B})₂—O—C(═O)R^{11B}. In some embodiments, when R^{5B} is C(R^{10B})₂—O—C(═O)R^{11B} then both R^{10B} can be hydrogen and R^{11B} can be an optionally substituted C₁₋₄ alkyl. In an embodiment, R^{5B} can be methyl. In another embodiment, R^{5B} can be tert-butyl.

In an embodiment, the compound of Formulae (I) and/or (Ia) can be selected from the following:

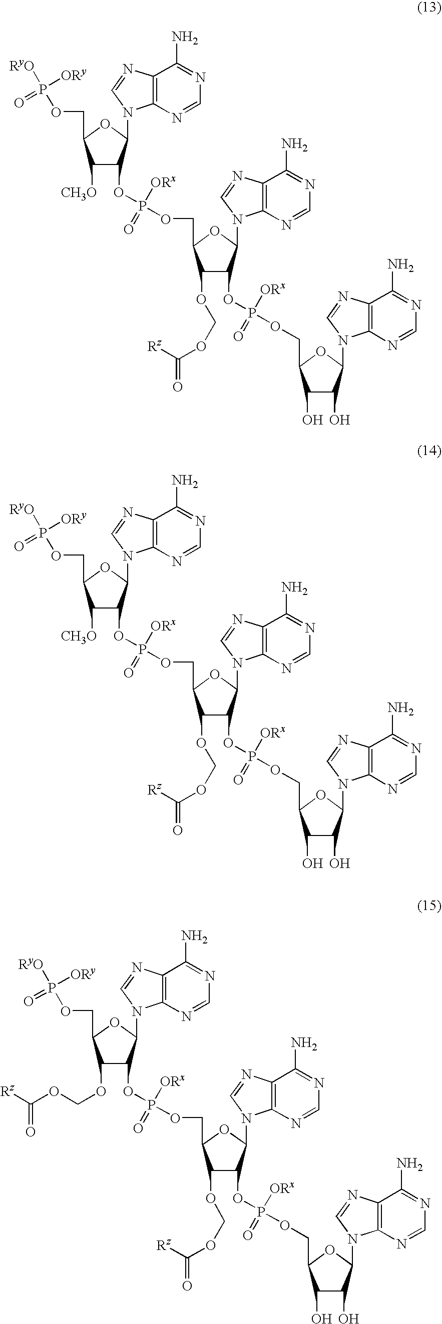

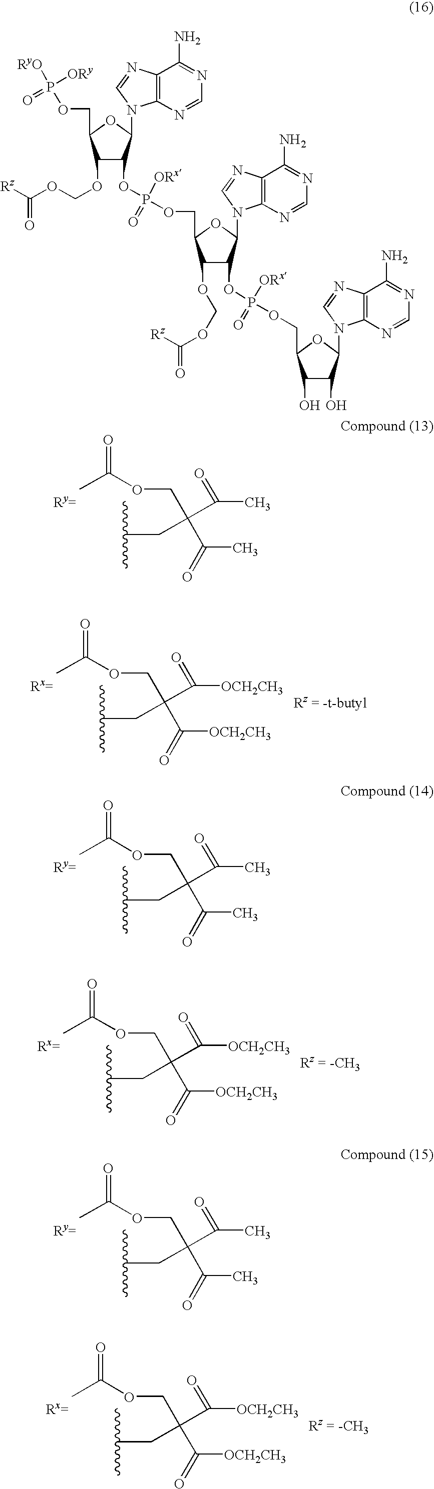

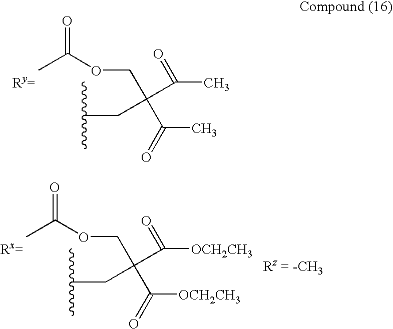

Without asking to be bound by any particular theory, it is believed that neutralizing the charge on one or more of the phosphate groups facilitates the penetration of the cell membrane by compounds of Formulae (I) and (Ia) by making the compound more lipophilic. Furthermore, it is believed that the 2,2-disubstituted-acyl(oxyalkyl) groups; for example

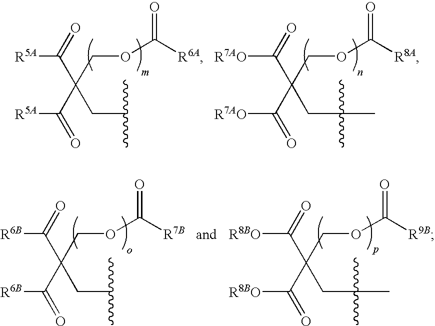

attached to the phosphate impart increased plasma stability to the compounds of Formulae (I) and (Ia) by inhibiting the degradation of the compound. Once inside the cell, the 2,2-disubstituted-acyl(oxyalkyl) groups attached to the phosphate can be easily removed by esterases via enzymatic hydrolysis of the acyl group. The remaining portions of the group on the phosphate can then be removed by elimination. The general reaction scheme is shown below in Scheme 1. Upon removal of the 2,2-disubstituted-acyl(oxyalkyl) group, the resulting nucleotide analog possesses a monophosphate. Thus, in contrast to use of trinucleoside compounds, the necessity of an initial intracellular phosphorylation is no longer a prerequisite to obtaining the biologically active phosphorylated form.

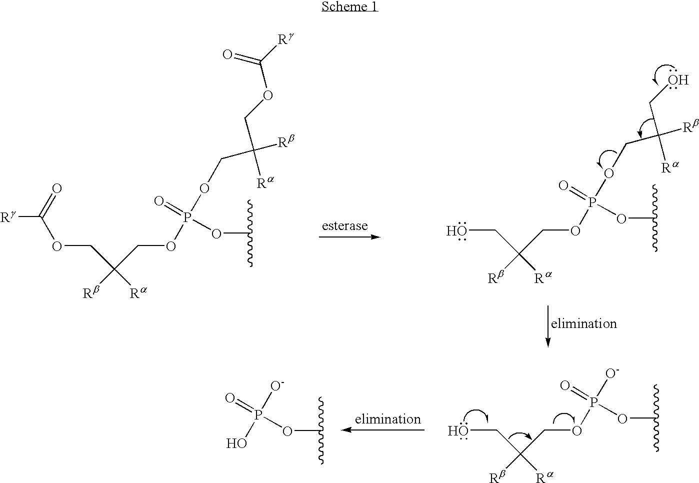

A further advantage of the 2,2-disubstituted-acyl(oxyalkyl) groups described herein is the rate of elimination of the remaining portion of the 2,2-disubstituted-acyl(oxyalkyl) group is modifiable. Depending upon the identity of the groups attached to the 2-carbon, shown in Scheme I as R^{α} and R^{β}, the rate of elimination may be adjusted from several seconds to several hours. As a result, the removal of the remaining portion of the 2,2-disubstituted-acyl(oxyalkyl) group can be retarded, if necessary, to enhance cellular uptake but, readily eliminated upon entry into the cell.

Additionally, when groups on the 2-carbon are identical, the 2,2-disubstituted-acyl(oxyalkyl) group is achiral, thus, markedly reducing the number of stereoisomers in the final compound (e.g., compounds of Formulae (I) and (Ia)). Having achiral 2,2-disubstituted-acyl(oxyalkyl) group also can simplify separation and characterization of the trimers.

When the group on the 3′-position on the middle residue is protected with an acyloxyalkyl group, it can also be removed by esterases via enzymatic hydrolysis of the acyl group followed by elimination of the remaining portion of the group. By varying the group at the 3′-position of the middle residue, the rate of elimination can be modified. It is believed that protecting the 3′-position minimizes and/or inhibits the isomerization of the phosphate on the 2′-position to the 3′-position. Additionally, protection of the 3′-position can reduce the likelihood that the phosphate will be prematurely cleaved off before entry into the cell.

Similarly, when the 3′-position of the 5′-terminal residue is protected, isomerization and premature cleavage of the neighboring 2′-phosphate can be minimized and/or inhibited. Also, when the 3′-position on the 5′-terminal residue is protected, the rate of removal can be modified similarly as discussed above with respect to the 3′-position on the middle residue.

As noted above, the rate of elimination of the groups on the 3′-positions and the phosphates can be adjusted; thus, in some embodiments, the identity of the groups on the phosphates and the 3′-positions can be chosen such that one or more groups on the phosphates are removed before the groups on the 3′-positions. In other embodiments, the identity of the groups on the phosphates and the 3′-positions can be chosen such that at least one group on the phosphates is removed after the groups on the 3′-positions. In an embodiment, the identity of the groups on the phosphates and the 3′-positions can be chosen such that the groups on the internal phosphates attached to the middle and 2′-terminal residues are removed before the groups on the 3′-positions of the middle and 5′-terminal residues. In another embodiment, the identity of the groups on the phosphates and the 3′-positions can be chosen such that the groups on the internal phosphates attached to the middle and 2′-terminal residues are removed before at least one group on the 5′-terminal phosphate and at least one group on the 5′-terminal residue is removed before the groups on the 3′-positions of the middle and 5′-terminal residues. In still another embodiment, the identity of the groups on the phosphates and the 3′-positions can be chosen such that the groups on the internal phosphates attached to the middle and 2′-terminal residues are removed before the groups on the 5′-terminal phosphate which in turn are removed before the groups on the 3′-positions of the middle and 5′-terminal residues.

While not wanting to be bound by any particular theory, it is believed that by protecting the phosphate groups and the 3′-positions of the middle and 5′-terminal residues, the breakdown of the trimer can be adjusted. This in turn can enhance cellular uptake and assist in maintaining the balance between unwanted viral RNA and native cellular RNA.

### Synthesis

Compounds of Formulae (I) and (Ia), and those described herein may be prepared in various ways. General synthetic routes to the compounds of Formulae (I) and (Ia) and the starting materials used to synthesize the compounds of Formula (I) and (Ia) are shown in Schemes 2a-2i. The routes shown are illustrative only and are not intended, nor are they to be construed, to limit the scope of the claims in any manner whatsoever. Those skilled in the art will be able to recognize modifications of the disclosed synthesis and to devise alternate routes based on the disclosures herein; all such modifications and alternate routes are within the scope of the claims.

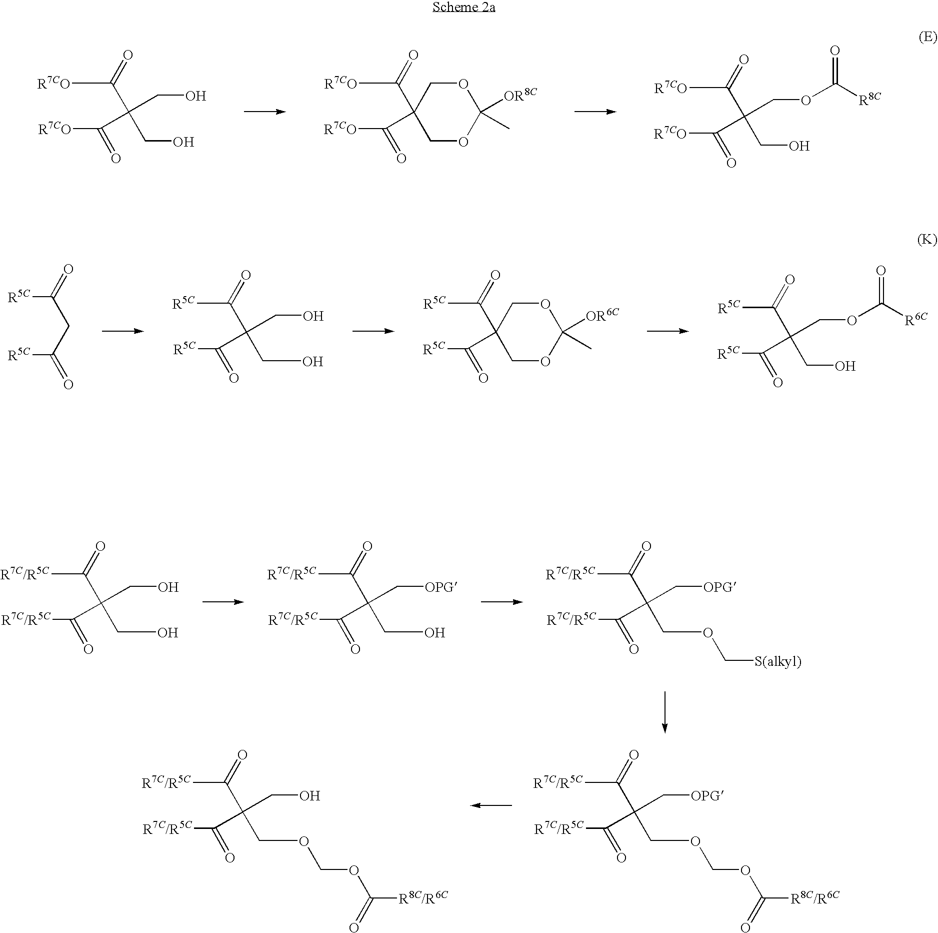

Compounds of Formulae E and K in which q and r are 1 can be synthesized as shown in above in Scheme 2a. For a compound of Formula E, an orthoester can be formed starting with a dialkyl-2,2-bis(hydroxymethyl)malonate. The ring of the orthoester can then be opened, using for example an acid, to form a compound of Formula E. Similarly, a compound of Formula K can be synthesized starting with an appropriate dione. Using an aldol condensation reaction, the dione can be transformed to a 2,2-bis(hydroxymethyl) dione. An orthoester can be formed from the 2,2-bis(hydroxymethyl) dione, followed by a ring-opening reaction to give a compound of Formula K. Compounds of Formulae W and CC in which s and t are one shown below can be synthesized in manners similar to those described above with respect to compounds of Formulae E and K.

Compounds of Formulae E, K, W and CC in which q, r, s and t are 2 can be synthesized starting with a dialkyl-2,2-bis(hydroxymethyl)malonate. One of the hydroxy groups can be protected with a suitable protecting group such as a silyl ether group. Suitable silyl ether groups are described herein. A methythiomethyl ether can be formed at the position occupied by the remaining hydroxyl group using acetic anhydride and dimethylsulfoxide (DMSO). The newly formed methythiomethyl ether can under to an oxidative-halogenation reaction using a suitable reagent such as sulfuryl chloride. An ester salt, such as potassium acetate, can then be added to form the terminal ester group. The protecting group on the initially protected hydroxyl group can be removed using a suitable reagent known to those skilled in the art, for example, an acid or tetraalkylammonium halide. The following articles provide exemplary methods for synthesizing the hydroxy precursors, compounds of Formulae E, K, W and CC: Ora, et al., J. Chem. Soc. Perkin Trans. 2, 2001, 6, 881-5; Poijärvi, P. et al., Helv. Chim. Acta. 2002, 85, 1859-76; Poijärvi, P. et al., Lett. Org. Chem., 2004, 1, 183-88; and Poijärvi, P. et al., Bioconjugate Chem., 2005 16(6), 1564-71, all of which are hereby incorporated by reference in their entireties.

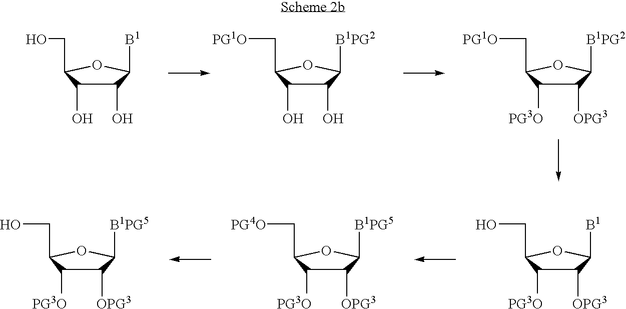

One example for synthesizing a compound that can be used to form the 2′-terminal residue is shown in Scheme 2b. The oxygen attached to the 5′-carbon and one or more amino groups attached to B¹ and/or a NH group(s) present in a ring of the heterocyclic base or derivative thereof, represented by B¹, can be protected using appropriate protecting group moieties represented by PG¹ and PG², respectively. If more then one amino group is attached to a heterocyclic base and/or derivative thereof, more than one protecting group can be used. If more than one protecting group is used, the protecting groups can be the same or different. In some embodiments, PG¹ and PG² can be the same or different. In an embodiment, PG¹ and PG² can be triarylmethyl protecting groups. A non-limiting list of triarylmethyl protecting groups are trityl, monomethoxytrityl (MMTr), 4,4′-dimethoxytrityl (DMTr), 4,4′,4″-trimethoxytrityl (TMTr), 4,4′,4″-tris-(benzoyloxy) trityl (TBTr), 4,4′,4″-tris (4,5-dichlorophthalimido) trityl (CPTr), 4,4′,4″-tris (levulinyloxy) trityl (TLTr), p-anisyl-1-naphthylphenylmethyl, di-o-anisyl-1-naphthylmethyl, p-tolyldiphenylmethyl, 3-(imidazolylmethyl)-4,4′-dimethoxytrityl, 9-phenylxanthen-9-yl (Pixyl), 9-(p-methoxyphenyl) xanthen-9-yl (Mox), 4-decyloxytrityl, 4-hexadecyloxytrityl, 4,4′-dioctadecyltrityl, 9-(4-octadecyloxyphenyl) xanthen-9-yl, 1,1′-bis-(4-methoxyphenyl)-1′-pyrenylmethyl, 4,4′,4″-tris-(tert-butylphenyl)methyl (TTTr) and 4,4′-di-3,5-hexadienoxytrityl.

Any oxygens attached as hydroxy groups to the 2′ and 3′-positions can also be protected using appropriate protecting groups. In some embodiments, the protecting groups on the 2′ and 3′-positions, represented by PG³, can be the same or different. In an embodiment, the PG³ groups are the same. In some embodiments, one or both PG³ groups can be silyl ether groups. Exemplary silyl ethers include, but are not limited to, trimethylsilyl (TMS), tert-butyldimethylsilyl (TBDMS), triisopropylsilyl (TIPS) and tert-butyldiphenylsilyl (TBDPS). In other embodiments, one or both PG³ groups can be levulinoyl groups.

After protecting any oxygens at the 2′ and 3′-positions, the protecting group on oxygen attached to the 5′-carbon and any protecting groups on the heterocyclic base can be removed. In some embodiments, the protecting groups on the oxygen attached to the 5′-carbon and any protecting groups on the heterocyclic base or heterocyclic base derivative can be removed using an acid (e.g., acetic acid). In an embodiment, the protecting group on the oxygen attached to the 5′-carbon can be removed before deprotecting one or more amino groups attached to B¹ and/or a NH group(s) present in a ring of B¹. In another embodiment, the protecting group on the oxygen attached to the 5′-carbon can be removed after deprotecting one or more amino groups attached to B¹ and/or a NH group(s) present in a ring of B¹. In still another embodiment, the protecting group on the oxygen attached to the 5′-carbon can be removed almost simultaneously with the removal of any protecting groups on the heterocyclic base or heterocyclic base derivative.

The oxygen attached to the 5′-carbon and one or more amino groups attached to B¹ and/or a NH group(s) present in a ring of the heterocyclic base or heterocyclic base derivative can then be reprotected using appropriate protecting groups represented by PG⁴ and PG⁵. The protecting groups PG⁴ and PG⁵ can be the same or different from the protecting groups used previously. In some embodiments, PG⁴ can be different from PG¹. In some embodiments, PG⁵ can be the same as PG². In an embodiment, the oxygen attached to the 5′-carbon can be protected with a silyl ether protecting group. As noted above, PG³, PG⁴ and PG⁵ can be different, thus, in some embodiments, PG³, PG⁴ and PG⁵ can be chosen such that conditions that would remove one of the group of PG³, PG⁴ and PG⁵ would not remove the remaining two protecting groups. As an example, PG³, PG⁴ and PG⁵ can be chosen such that PG⁵ can be removed without removing PG³ and/PG⁴. In some embodiments, one or more amino groups attached to B¹ and/or a NH group(s) present in a ring of the heterocyclic base can be protected with a triarylmethyl protecting group(s). In an embodiment, the oxygen attached to the 5′-carbon can be reprotected before reprotecting any amino groups attached to B¹ and/or a NH group(s) present in a ring of B¹. In other embodiments, any amino groups attached to B¹ and/or a NH group(s) present in a ring of B¹ can be reprotected before protecting the oxygen attached to the 5′-carbon.

In some embodiments, the oxygen attached to the 5′-carbon can then selectively deprotected using methods known to those skilled in the art. For example, the protecting group on the oxygen attached to the 5′-carbon can be selectively deprotected without removing any protecting groups on the heterocyclic base or heterocyclic base derivative and/or any protecting groups on the oxygens attached to the 2′ and 3′-positions. In an embodiment, the protecting group on the oxygen attached to the 5′-carbon can be removed with a tetraalkylammonium halide, such as tetra(t-butyl)ammonium fluoride, or an acid.

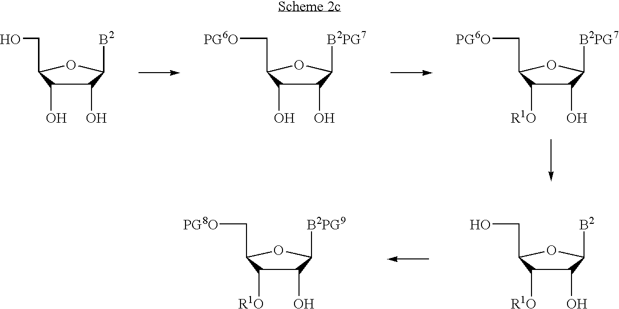

One example for synthesizing a nucleoside analog in which the 3′-position has R¹ being —C(R²)₂—O—C(═O)R³, wherein each R² and R³ are each independently hydrogen or an optionally substituted C₁₋₄ alkyl is shown in Scheme 2c. The oxygen attached to the 5′-carbon and one or more amino groups attached to B² and/or a NH group(s) present in a ring of the heterocyclic base or heterocyclic base derivative represented B² can be protected using appropriate protecting groups represented by PG⁶ and PG⁷, respectively. In some embodiments, PG⁶ and PG⁷ can be the same or different. In an embodiment, PG⁶ and PG⁷ can be triarylmethyl protecting groups. R¹ can be added by removing the hydrogen on the oxygen attached to the 3′-position using an appropriate reagent such as sodium hydride and adding the —C(R²)₂—O—C(═O)R³ group. In an embodiment, the —C(R²)₂—O—C(═O)R group can be added using an appropriate alkylating reagent, such as sodium iodide, and X¹—C(R²)₂—O—C(═O)R³, wherein R² and R³ are described herein and X¹ can be a halide. The protecting groups on the oxygen attached to the 5′-carbon and any protecting groups on the heterocyclic base or heterocyclic base derivative can then be removed using methods known to those in the art. For example, when PG⁶ and PG⁷ are triarylmethyl groups, both can be removed using an appropriate acid or a zinc dihalide (e.g., ZnBr₂). In some embodiments, the protecting groups on the oxygen attached to the 5′-carbon and any protecting groups on the heterocyclic base or heterocyclic base derivative can be removed using acetic acid. In an embodiment, the protecting group on the oxygen attached to the 5′-carbon can be removed before deprotecting one or more amino groups attached to B² and/or a NH group(s) present in a ring of B². In another embodiment, the protecting group on the oxygen attached to the 5′-carbon can be removed after deprotecting one or more amino groups attached to B² and/or a NH group(s) present in a ring of B². In still another embodiment, the protecting group on the oxygen attached to the 5′-carbon can be removed almost simultaneously with the removal of any protecting groups on the heterocyclic base or heterocyclic base derivative.

The oxygen attached to the 5′-carbon can then be reprotected with the same or different protecting groups as used previously. Similarly, any amino groups attached B² and/or a NH group(s) present in a ring of B² can be reprotected using the same or different protecting group as used previously. In some embodiments, PG⁸ and PG⁹ can be different. In some embodiments, PG⁸ can be different from PG⁶. In some embodiments, PG⁷ can be the same as PG⁹. In some embodiments, the oxygen attached to the 5′-carbon can be protected with a triarylmethyl group. In some embodiments, one or more amino groups attached to B² and/or a NH group(s) present in a ring of B² can be protected with a silyl ether group(s). In an embodiment, the oxygen attached to the 5′-carbon can be reprotected before reprotecting any amino groups attached to B² and/or a NH group(s) present in a ring of B². In other embodiments, any amino groups attached to B² and/or a NH group(s) present in a ring of B² can be reprotected before protecting the oxygen attached to the 5′-carbon. In an embodiment, PG⁸ can be a protecting group that cannot be removed under the same conditions as PG⁹. For example, PG⁹ can be a protecting group that can be removed by an acid that cannot remove PG⁸.

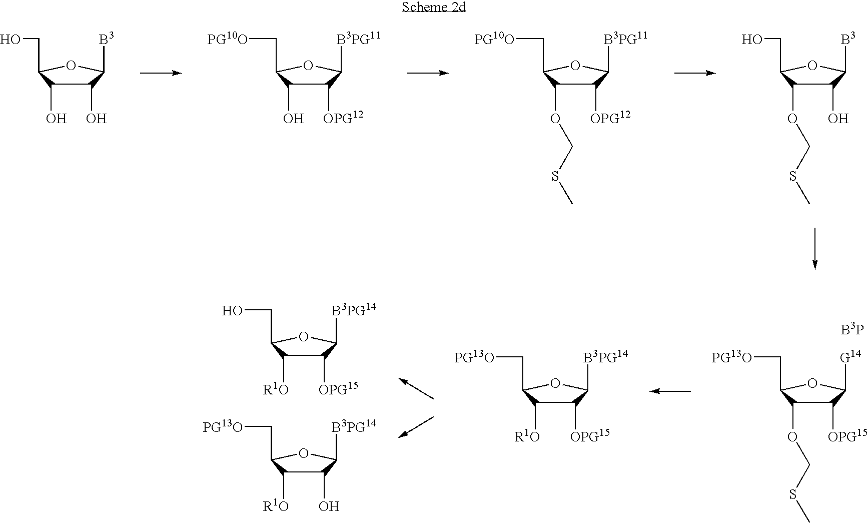

Another example for synthesizing a nucleoside analog in which the 3′-position has R¹ being —C(R²)₂—O—C(═O)R³, wherein each R² and R³ are each independently hydrogen or an optionally substituted C₁₋₄-alkyl is shown in Scheme 2d. The oxygen attached to the 5′-carbon, any amino groups attached to B³ and/or a NH group(s) present in a ring of the heterocyclic base or heterocyclic base derivative represented by B³ and any oxygens attached as hydroxy groups to the 2′-position can be protecting using appropriate protecting groups represented by PG¹⁰, PG¹¹ and PG¹². In some embodiments, one, two or all of PG¹⁰, PG¹¹ and PG¹² can be the same or different. In an embodiment, PG¹⁰, PG¹¹ and PG¹² can be triarylmethyl protecting groups. The hydrogen of the —OH group attached to the 3′-position can then be removed using methods known to those skilled in the art, such as sodium hydride, followed by alkylation with a (halomethyl)(alkyl)sulfane. Any protecting groups represented by PG¹⁰, PG¹¹ and PG¹² can be then removed using methods known to those skilled in the art. For example, when PG¹⁰, PG¹¹ and PG¹² are triarylmethyl groups, PG¹⁰, PG¹¹ and PG¹² can be removed using an acid such as acetic acid or a zinc dihalide such as zinc dibromide. In an embodiment, PG¹⁰, PG¹¹ and PG¹² can be removed with acetic acid.

The oxygen attached to the 5′-carbon, any amino groups attached to B³ and/or a NH group(s) present in a ring of B³ and any oxygens attached as hydroxy groups to the 2′-position can be reprotected using appropriate protecting groups which can be the same of different from those used previously. In some embodiments, PG¹³ can be different from PG¹⁰. In an embodiment, PG¹⁴ can be the same as PG¹¹. In some embodiments, PG¹⁵ can be different from PG². In other embodiments, PG¹⁵ can be the same as PG¹². In some embodiments, the oxygen attached to the 5′-carbon can be protected using a triarylmethyl protecting group. In an embodiment, any amino groups attached to B³ and/or a NH group(s) present in a ring of B³ can be protected with a silyl ether group(s). In some embodiments, any oxygens attached as hydroxy groups at the 2′-position can be protected using levulinoyl group(s). In other embodiments, any oxygens attached as hydroxy groups to the 2′-position can be protected using silyl ether group(s). In an embodiment, PG¹³, PG¹⁴ and PG¹⁵ can be different from each other. In an embodiment, the oxygen attached to the 5′-carbon can be reprotected before reprotecting any amino groups attached to B³ and/or a NH group(s) present in a ring of B³ and/or any oxygens attached as hydroxy groups to the 2′-position. In some embodiments, any amino groups attached to B³ and/or a NH group(s) present in a ring of B³ can be reprotected after protecting the oxygen attached to the 5′-carbon but before reprotecting any oxygens attached as hydroxy groups to the 2′-position. In an embodiment, any oxygens attached as hydroxy groups to the 2′-position can be reprotected after reprotecting the oxygen attached to the 5′-carbon and any amino groups attached to B³ and/or a NH group(s) present in a ring of B³. In some embodiments, PG¹³ can be a protecting group that can be selectively removed without removing PG¹⁴ and/or PG¹⁵. As example, PG¹³ can be a protecting group that can be removed using a tetraalkylammonium halide that cannot remove PG¹⁴ and/or PG¹⁵. In an embodiment, PG¹⁴ can be a protecting group that cannot be removed under the same conditions as PG¹³ and/or PG¹⁵. For example, PG¹⁴ can be a protecting group that cannot be removed by a tetraalkylammonium halide or hydrazinium acetate when one or either condition can remove PG¹³ and/or PG¹⁵. In some embodiments, PG¹⁵ can be a protecting group than cannot be removed under the same conditions as PG¹³ and/or PG¹⁴. For example, PG¹⁵ can be levulinoyl group that can be removed using hydrazinium acetate which cannot remove PG¹³ and/or PG¹⁴. In other embodiments, PG¹⁴ and PG¹⁵ can be removed under the same conditions, but those conditions cannot remove PG¹³.

The methyl(alkyl)sulfane added to the oxygen attached to the 2′-position can under go an oxidative-halogenation reaction using an appropriate reagent such as sulfuryl chloride. An ester in form of an ester salt can then be added to form R¹. The protecting groups, PG¹³ can then be selectively removed. For example, as described above PG¹³ can be removed without removing PG¹⁴ and/or PG¹⁵. In an embodiment, PG¹³ can be removed using a tetraalkylammonium halide such as tetrabutylammonium fluoride. In another embodiment, PG¹⁵ can be selectively removed such that PG¹⁵ is removed without removing PG¹³ and/or PG¹⁴. In an embodiment, PG¹⁵ can be removed with hydrazinium acetate.

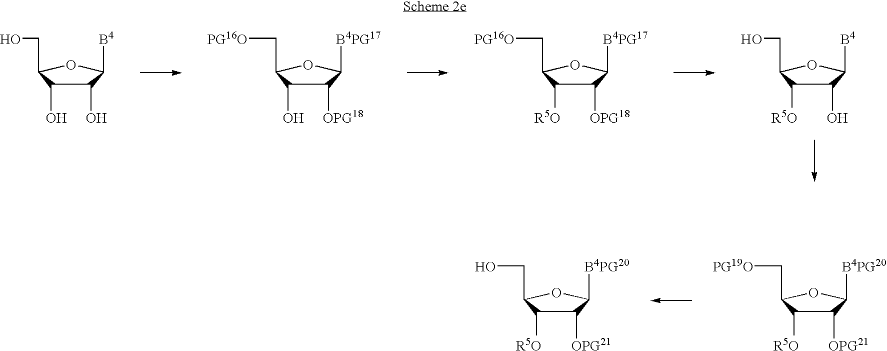

An example for synthesizing a nucleoside analog in which the substituent attached to the 3′-position has R⁵ being —OR⁵ in which R⁵ is an optionally substituted C₁₋₄-alkyl is shown in Scheme 2e. The oxygen attached to the 5′-carbon, any amino groups attached to the heterocyclic base or heterocyclic base derivative represented by B⁴ and any oxygens attached as hydroxy groups to the 2′-position can be protecting using appropriate protecting groups represented by PG¹⁶, PG¹⁷ and PG¹⁸. In some embodiments, one, two or all of PG¹⁶, PG¹⁷ and PG¹⁸ can be the same or different. In an embodiment, PG¹⁶, PG¹⁷ and PG¹⁸ can be triarylmethyl protecting groups. The hydrogen of the —OH attached to the 3′-position can then be removed using methods known to those skilled in the art such as sodium hydride followed by alkylation with a haloalkyl, which can be optionally substituted. Any protecting groups represented by PG¹⁶, PG¹⁷ and PG¹⁸ can be then removed using the appropriate reagent and conditions known to those skilled in the art. For example, when PG¹⁶, PG¹⁷ and PG¹⁸ can be removed using an acid or a zinc dihalide. In an embodiment, PG¹⁶, PG¹⁷ and PG¹⁸ can be removing using acetic acid.

The oxygen attached to the 5′-carbon, any amino groups attached to B⁴ and/or a NH group(s) present in a ring of B⁴ and any oxygens attached as hydroxy groups to the 2′-position can be reprotected using appropriate protecting groups which can be the same or different from those protecting groups used previously. In some embodiments, PG¹⁹ can be different from PG¹⁶. In an embodiment PG²⁰ can be different from PG¹⁷. In some embodiments, PG²¹ can be different from PG¹⁸. In other embodiments, PG²¹ can be the same as PG¹⁸. In some embodiments, the oxygen attached to the 5′-carbon can be protected using a triarylmethyl protecting group. In an embodiment, any amino groups attached to the heterocyclic base or heterocyclic base derivative can be protected with a silyl ether group(s). In some embodiments, any oxygens attached as hydroxy groups to the 2′-position can be protected using levulinoyl group(s). In other embodiments, any oxygens attached as hydroxy groups to the 2′-position can be protected using silyl group(s). In an embodiment, PG¹⁹, PG²⁰ and PG²¹ can be different from each other. In an embodiment, the oxygen attached to the 5′-carbon can be reprotected before reprotecting any amino groups attached to B⁴ and/or a NH group(s) present in a ring of B⁴ and/or any oxygens attached as hydroxy groups to the 2′-position. In some embodiments, any amino groups attached to B⁴ and/or a NH group(s) present in a ring of B⁴ can be reprotected after protecting the oxygen attached to the 5′-carbon but before reprotecting any oxygens attached as hydroxy groups to the 2′-position. In an embodiment, any oxygens attached as hydroxy groups to the 2′-position can be reprotected after reprotecting the oxygen attached to the 5′-carbon and any amino groups attached to B⁴ and/or a NH group(s) present in a ring of B⁴. In an embodiment, PG¹⁹ can be a protecting group that can be selectively removed without removing PG²⁰ and/or PG²¹. As example, PG¹⁹ can be a protecting group that can be removed using a tetraalkylammonium halide that cannot remove PG²⁰ and/or PG²¹. In an embodiment, PG²⁰ can be a protecting group that cannot be removed under the same conditions as PG¹⁹ and/or PG²¹. For example, PG can be a protecting group that cannot be removed by a tetraalkylammonium halide or hydrazinium acetate when one or either condition can remove PG¹⁹ and/or PG²¹. In some embodiments, PG²¹ can be a protecting group than cannot be removed under the same conditions as PG¹⁹ and/or PG 20. For example, PG²¹ can be levulinoyl group that can be removed using hydrazinium acetate which cannot remove PG²⁰ and/or PG²¹.

The protecting groups, PG¹⁹ can be selectively removed. As described above, PG¹⁹ can be chosen such that it can be removed without removing PG²⁰ and/or PG²¹. In an embodiment, PG¹⁹ can be removed using a tetraalkylammonium halide such as tetrabutylammonium fluoride.

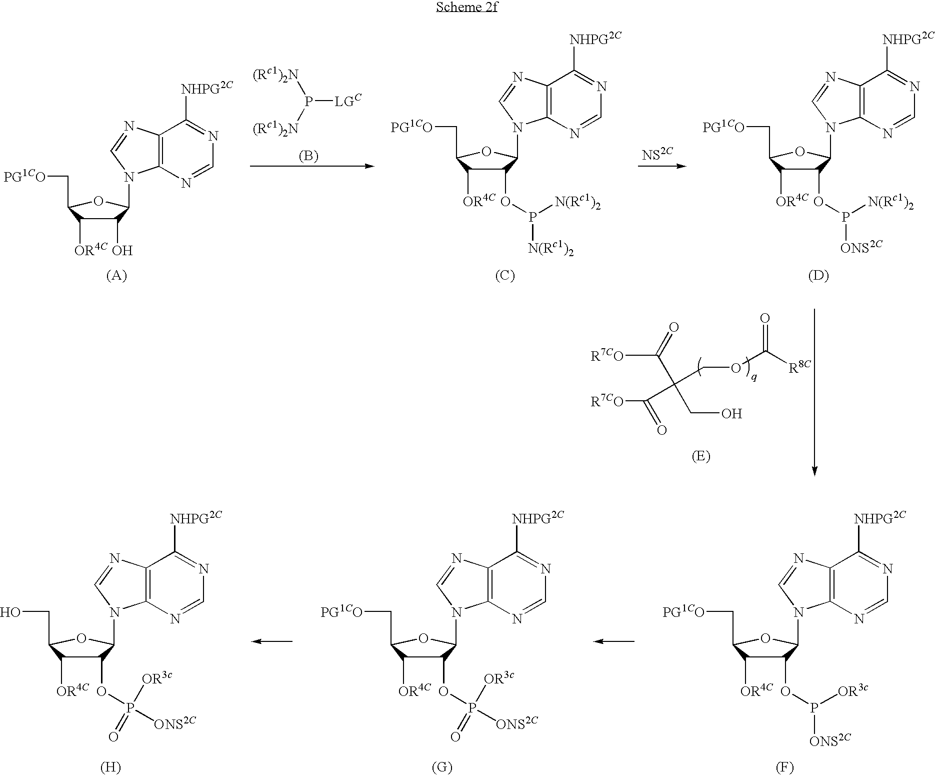

One embodiment disclosed herein relates to a method of synthesizing a compound of Formula H that includes the transformations shown in Scheme 2f. In Scheme 2f, R^{3C}, R^{4C}, R^{7C}R^{8C} NS^{2C} and q can be the same as R^{3A}, R^{4A}, R^{7A}, R^{8A} NS^{2A} and n, respectively, as described above with respect Formula (I). PG^{1C} and PG^{2C} represent appropriate protecting groups. In some embodiments, PG^{1C} can be a silyl ether. Exemplary silyl ethers are described above. In an embodiment of the method shown in Scheme 2f, PG^{2C} can be a triarylmethyl protecting group. Examples of suitable triarylmethyl protecting groups are described herein.

A compound of Formula C can be produced by forming a phosphoamidite at the 2′-position of a compound of Formula A by reacting a compound of Formula B with the —OH attached to the 2′-position of a compound of Formula A to form a compound of Formula C. In an embodiment, each R^{C1} can be independently an optionally substituted C₁₋₄ alkyl, and LG^{C} can be a suitable leaving group. In an embodiment, the leaving group on a compound of Formula B can be a halogen. One benefit of having the other hydroxy groups and any amino groups attached to the heterocyclic base or derivative thereof and/or a NH group(s) present in a ring of the heterocyclic base or derivative thereof protected is that the addition of a compound of Formula B can be directed to the 2′-position of a compound of Formula A. Furthermore, the protecting groups on the hydroxy groups and any amino groups attached to the heterocyclic base or derivative thereof and/or a NH group(s) present in a ring of the heterocyclic base or derivative thereof can block undesirable side reactions that may occur during later synthetic transformations. Minimization of unwanted side compound can assist in the separation and isolation of the desired compound(s).

A nucleoside, a nucleoside analog, a protected nucleoside or a protected nucleoside analog can be added to a compound of Formula C in which the —OH attached to the 5′-carbon group of the nucleoside, a nucleoside analog, a protected nucleoside or a protected nucleoside analog reacts with the phosphoamidite of a compound of Formula C to form a compound of Formula D. In some embodiments, the nucleoside, the nucleoside analog, the protected nucleoside or the protected nucleoside analog can have the structure of a compound of Formula LL,

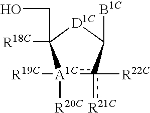

in which [symbol] can be a double or single bond; A^{1C} can be selected from C (carbon), O (oxygen) and S (sulfur); B^{1C} can be selected from an optionally substituted heterocyclic base, an optionally substituted heterocyclic base derivative, an optionally substituted protected heterocyclic base, and an optionally substituted protected heterocyclic base derivative; D^{1C} can be C═CH₂ or O (oxygen); R^{18C} can be selected from hydrogen, azido, —CN, an optionally substituted C₁₋₄ alkyl and an optionally substituted C₁₋₄ alkoxy; R^{19C} can be absent or selected from hydrogen, halogen, hydroxy and an optionally substituted C₁₋₄ alkyl; R^{20C} can be absent or selected from hydrogen, halogen, azido, amino, hydroxy and -OPG^{3C}; R^{21C} can be selected from hydrogen, halogen, hydroxy, —CN, —NC, an optionally substituted C₁₋₄ alkyl, an optionally substituted C₁₋₄ alkoxy and -OPG^{4C}; R^{22C} can be absent or selected from hydrogen, halogen, hydroxy, —CN, —NC, an optionally substituted C₁₋₄ alkyl, an optionally substituted haloalkyl and an optionally substituted hydroxyalkyl, or when the bond to R^{21C} indicated by [symbol] is a double bond, then R^{21C} is a C₁₋₄ alkenyl and R^{22C} is absent; and PG^{3C} and PG^{4C} can each be a protecting group. In some embodiments, PG^{3C} can be a levulinoyl group. In some embodiments, PG^{4C} can be a levulinoyl group. In other embodiments, PG^{3C} can be a silyl ether group. In other embodiments, PG^{4C} can be a silyl ether group.

To facilitate the reaction between the nucleoside, the nucleoside analog, the protected nucleoside or the protected nucleoside analog and a compound of Formula C, an activator can be used. An exemplary activator is a tetrazole such as benzylthiotetrazole. The tetrazole can protonate the nitrogen of the phosphoamidite making it susceptible to nucleophilic attack by the nucleoside or nucleoside analog. Additional activators that can be used are disclosed in Nurminen, et al., J. Phys. Org. Chem., 2004, 17, 1-17 and Michalski, J. et al., Stated of the Art. Chemical Synthesis of Biophosphates and their Analogues via P^{III} Derivatives, Springer Berlin (2004) vol. 232, pages 43-47; which is hereby incorporated by reference for the limited purpose of their disclosure of additional activators.

A R^{3C} moiety can be added to a compound of Formula D by reacting a compound of Formula D with a compound of Formula E to form a compound of Formula F. An activator can also be used to promote this reaction as described above. As mentioned previously, having protecting group(s) on the hydroxy groups and any amino groups attached to the heterocyclic base or derivative thereof and/or a NH group(s) present in a ring of the heterocyclic base or derivative thereof can direct the addition of compounds such as a compound of Formula E. As a result, undesirable side reactions that may occur during later synthetic transformations can be minimized, thus, making the separation and isolation of the desired compound(s) more facile.

The phosphite of a compound of Formula F can be oxidized to a phosphate moiety to form a compound of Formula G. In an embodiment, the oxidation can be carried out using iodine as the oxidizing agent and water as the oxygen donor.

The protecting group moiety, PG^{1C}, can be removed to form a compound of Formula H. In an embodiment, PG^{1C} can be removed with a tetra(alkyl)ammonium halide such as tetra(t-butyl)ammonium fluoride. In some embodiments, PG^{1C} can be selectively removed such that PG^{1C} is removed without removing PG^{2C}. For example, PG^{1C} can be removed using a reagent such as a tetra(alkyl)ammonium halide that does not remove PG^{2C}.

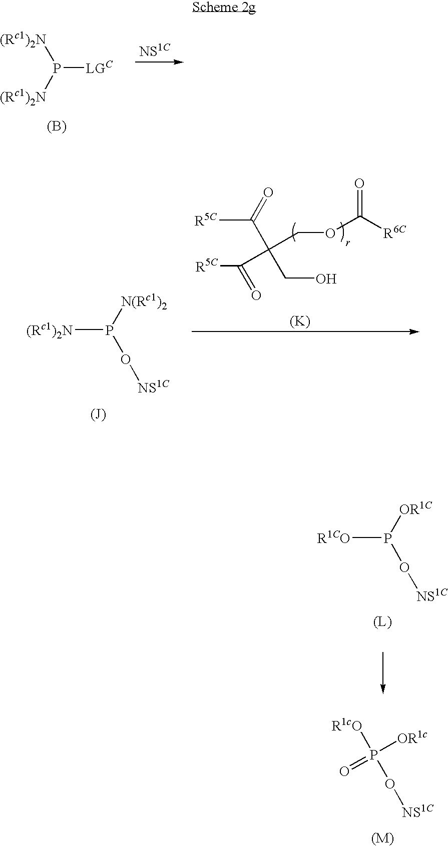

An embodiment disclosed herein relates to a method of synthesizing a compound of Formula M as shown in Scheme 2g. In Scheme 2g, R^{1C}, R^{5C}, R^{6C}, NS^{1C} and r can be the same as R^{1A}, R^{5A}, R^{6A}, NS^{1A} and m, respectively, as described above with respect Formula (I).

A phosphoamidite can be formed at the 5′-position or equivalent position of a nucleoside, a nucleoside analog, a protected nucleoside or a protected nucleoside analog by reacting a compound of Formula B with NS^{1C} to form a compound of Formula J. In an embodiment, each R^{C1} can be independently an optionally substituted C₁₋₄ alkyl, and LG^{C} can be a suitable leaving group. In some embodiments, the leaving group on a compound of Formula B can be a halogen.

In some embodiments, the nucleoside, the nucleoside analog, the protected nucleoside or the protected nucleoside analog being reacted with a compound of Formula B can have the structure of a compound of Formula MM,

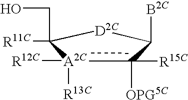

in which [symbol] can be a double or single bond; A^{2C} can be selected from C (carbon), O (oxygen) and S (sulfur); B^{2C} can be selected from an optionally substituted heterocyclic base, an optionally substituted heterocyclic base derivative, an optionally substituted protected heterocyclic base, and an optionally substituted protected heterocyclic base derivative; D^{2C} can be C═CH₂ or O (oxygen); R^{11C} can be selected from hydrogen, azido, —CN, an optionally substituted C₁₋₄ alkyl and an optionally substituted C₁₋₄ alkoxy; R^{12C} can be absent or selected from hydrogen, halogen, hydroxy and an optionally substituted C₁₋₄ alkyl; R^{13C} can be absent or selected from hydrogen, halogen, azido, amino, hydroxy, an optionally substituted C₁₋₄ alkoxy and OC(R^{16C})₂—O—C(═O)R^{17C}; R^{15C} can be absent or selected from hydrogen, halogen, hydroxy, —CN, —NC, an optionally substituted C₁₋₄ alkyl, an optionally substituted haloalkyl and an optionally substituted hydroxyalkyl; each R^{16C} and R^{17C} can be independently hydrogen or an optionally substituted C₁₋₄-alkyl; and PG^{5C} can be a protecting group. In some embodiments, PG^{5C} can be a levulinoyl group. In other embodiments, PG^{5C} can be a silyl ether group.

In an embodiment, B^{1C} and B^{2C} can each be independently selected from:

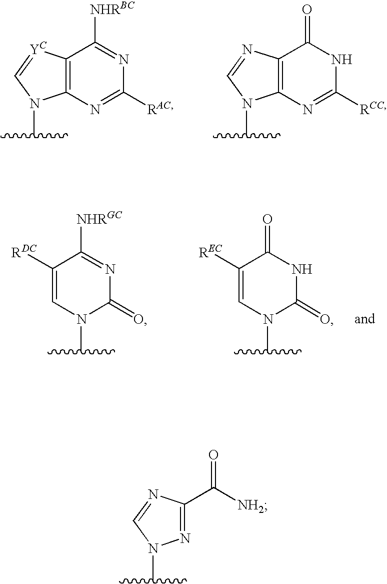

in which R^{AC} can be hydrogen or halogen; R^{BC} can be hydrogen, an optionally substituted C₁₋₄ alkyl, an optionally substituted C₃₋₈ cycloalkyl or a protecting group; R^{CC} can be hydrogen or amino; R^{DC} can be hydrogen or halogen; R^{EC} can be hydrogen or an optionally substituted C₁₋₄ alkyl; Y^{C} can be N (nitrogen) or CR^{FC}, wherein R^{FC} hydrogen, halogen or an optionally substituted C₁₋₄ alkyl; and R^{GC} can be a protecting group. In an embodiment, one or both of R^{BC} and R^{GC} can be a triarylmethyl protecting group such as those described previously. In an embodiment, B^{1C} and B^{2C} can be the same. In another embodiment, B^{1C} and B^{2C} can be different.

A R^{1C} moiety can be added to a compound of Formula J by reacting a compound of Formula K with a compound of Formula J to form a compound of Formula L. As shown above, the R^{1C} moiety can be added to the phosphorous to form a compound of Formula L. As described herein, an activator can be used to assist the addition.

A compound of Formula M can be obtained by oxidizing the phosphite to a phosphate using an appropriate oxidizing agent and oxygen donor. In an embodiment, the oxidizing agent can be iodine and the oxygen donor can be water.

In some embodiments, various protecting groups may be present on NS^{1C}. For example, any hydroxy groups attached to the 2′-position and 3′-position may be protected using one or more appropriate protecting groups, such as a levulinoyl group. Similarly, any amino groups and/or any —NH groups present in the ring of the heterocyclic base or heterocyclic base derivative may be protected using suitable one or more suitable protecting groups. Suitable protecting groups include, but are not limited to, silyl ethers and triarylmethyl groups. The protecting groups can promote the addition of a compound of Formula K to the 5′-position or equivalent position of NS^{1C}. Thus, the presence of protecting groups on NS^{1C} can be advantageous for minimizing unwanted side reactions. Additionally, by minimizing the number and/or amount of side products, the separation and isolation of the desired product can be made easier.

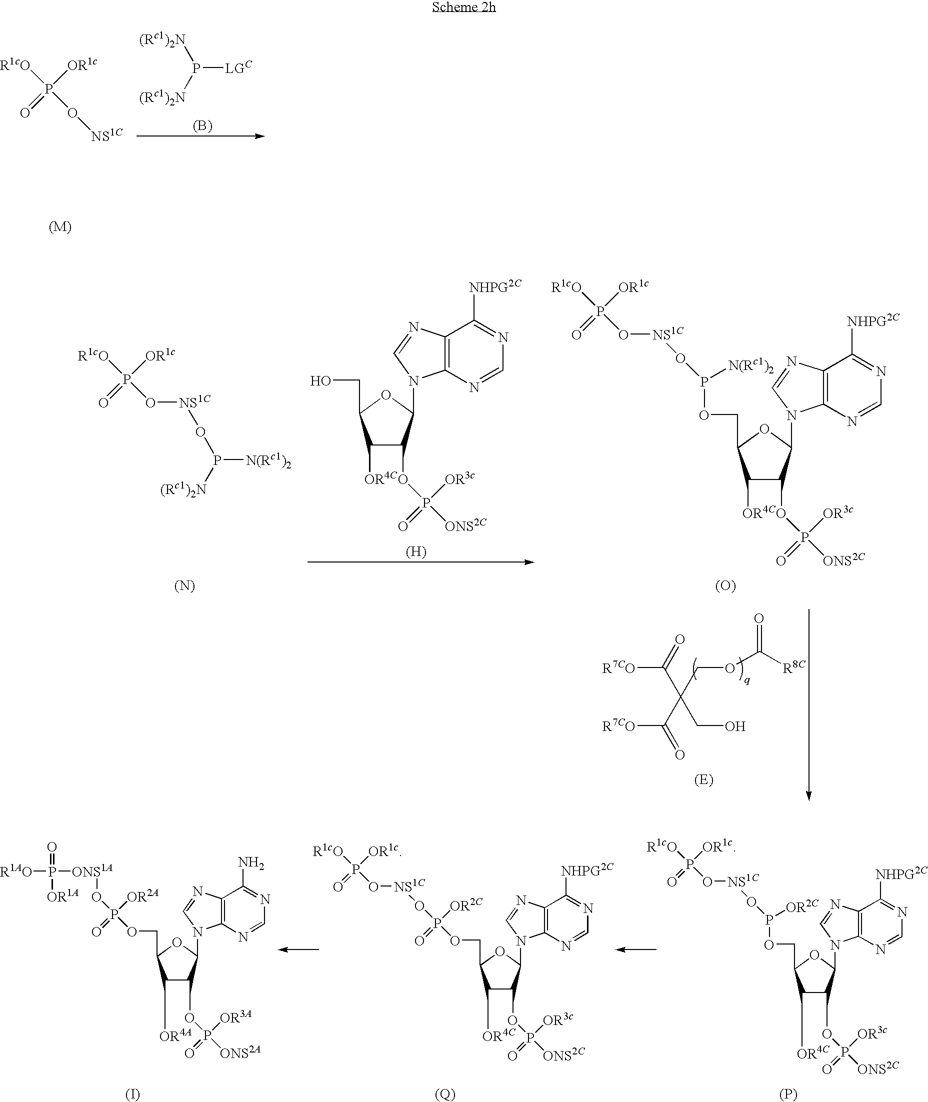

Some embodiments disclosed herein relate to a method of synthesizing a compound of Formula (I) as shown in Scheme 2h. In Scheme 2b, R^{1C}, R^{2C}, R³, R^{4C}, R^{5C}, R^{6C}, R^{7C}, R^{8C}, NS^{1C}, NS^{2C}, q and r can be the same as R^{1A}, R^{2A}, R^{3A}, R^{4A}, R^{5A}, R^{6A}, R^{7A}, R^{8A}, NS^{1A}, NS^{2A}, n and m, respectively, as described above with respect Formula (I). PG^{2C} represents an appropriate protecting group. In an embodiment, PG^{2C} can be a triarylmethyl protecting group. Exemplary triarylmethyl protecting groups are described herein.

A phosphoamidite can be formed on the nucleoside, the nucleoside analog, the protected nucleoside or the protected nucleoside analog represented by NS^{1C} by reacting a compound of Formula B with a compound of Formula M to form a compound of Formula N. In an embodiment, each R^{C1} can be independently an optionally substituted C₁₋₄ alkyl, and LG_{C} can be a suitable leaving group. In some embodiments, the leaving group on a compound of Formula B can be a halogen. In an embodiment, the phosphoamidite is formed at the 2′-position or equivalent position thereof of a nucleoside, a nucleoside analog, a protected nucleoside or a protected nucleoside analog.

A compound of Formula H that can be obtained from the synthetic route shown in Scheme 2f can be added to a compound of Formula N to form a compound of Formula O. In some embodiments, the —OH attached to the 5′-carbon on a compound of Formula H can be added to the phosphoamidite of a compound of Formula N to form a compound of Formula O. As described previously herein, an activator such as a tetrazole can be used to facilitate the addition.

A R^{2C} moiety can be added to a compound of Formula O by reacting a compound of Formula E with a compound of Formula O to form a compound of Formula P. As shown in Scheme 2h, the R^{2C} moiety can be added to the phosphorous of a compound of Formula O to form a compound of Formula P. The addition of a compound of Formula E and a compound of Formula O can be also assisted with an activator such as those described herein.

A compound of Formula Q can be obtained by oxidizing the phosphite of a compound of Formula P with an appropriate oxidizing agent and oxygen source. In an embodiment, the oxidizing agent can be iodine and the oxygen source can be water.

The protecting group represented by PG^{2C}, any additional protecting groups present attached to the heterocyclic bases or heterocyclic base derivatives of NS^{1C} and NS^{2C}, and any protecting group on the oxygens attached as hydroxy groups to the 2′ and 3′-positions of NS^{1C} and NS^{2C} can be removed using methods known to those skilled in the art to form a compound of Formula (I). In an embodiment, PG^{2C} can be removed with an acid such as acetic acid or a zinc dihalide, such as ZnBr₂. In some embodiments, the heterocyclic bases or heterocyclic base derivatives of NS^{1C} and NS^{2C} are protected with triarylmethyl protecting groups which can removed with an acid (e.g., acetic acid). In some embodiments, levulinoyl protecting groups can be attached to one or more oxygens of NS^{2C}. In an embodiment, the levulinoyl protecting groups can be removed with hydrazinium acetate. In other embodiment, silyl ether protecting groups can be attached to one or more oxygens of NS^{2C}. In an embodiment, the silyl ether groups can be removed using a tetraalkylammonium halide (e.g., tetrabutylammonium fluoride). In some embodiments, the protecting groups on the oxygens attached to the 2′ and 3′-positions of NS^{2C}, if present, can be removed selectively. For example, the groups on the oxygens attached to the 2′ and 3′-positions of NS^{2C} can be removed without removing any protecting groups attached to the heterocyclic bases or heterocyclic base derivatives of NS^{1C} and NS^{2C}. Alternatively, any protecting groups on the heterocyclic bases of NS^{1C} and NS^{2C} can be selectively removed such that the protecting groups on the heterocyclic bases or heterocyclic base derivatives of NS^{1C} and NS^{2C} can be removed without removing any protecting groups on the oxygens attached to the 2′ and 3′-positions of NS^{2C}. In an embodiment, the protecting groups on the oxygens attached to the 2′ and 3′-positions of NS^{2C}, if present, can be removed before removing any protecting groups on the heterocyclic bases or heterocyclic base derivatives of NS^{1C} and NS^{2C}. In another embodiment, the protecting groups on the oxygens attached to the 2′ and 3′-positions of NS^{2C}, if present, can be removed after removing any protecting groups on the heterocyclic bases or heterocyclic base derivatives of NS^{1C} and NS^{2C}.

In Schemes 2f, 2g and 2h, the compounds of Formula B used form the phosphoamidites can be the same or different. Similarly, the compounds of Formula E in Schemes 2f and 2h can be the same or different.

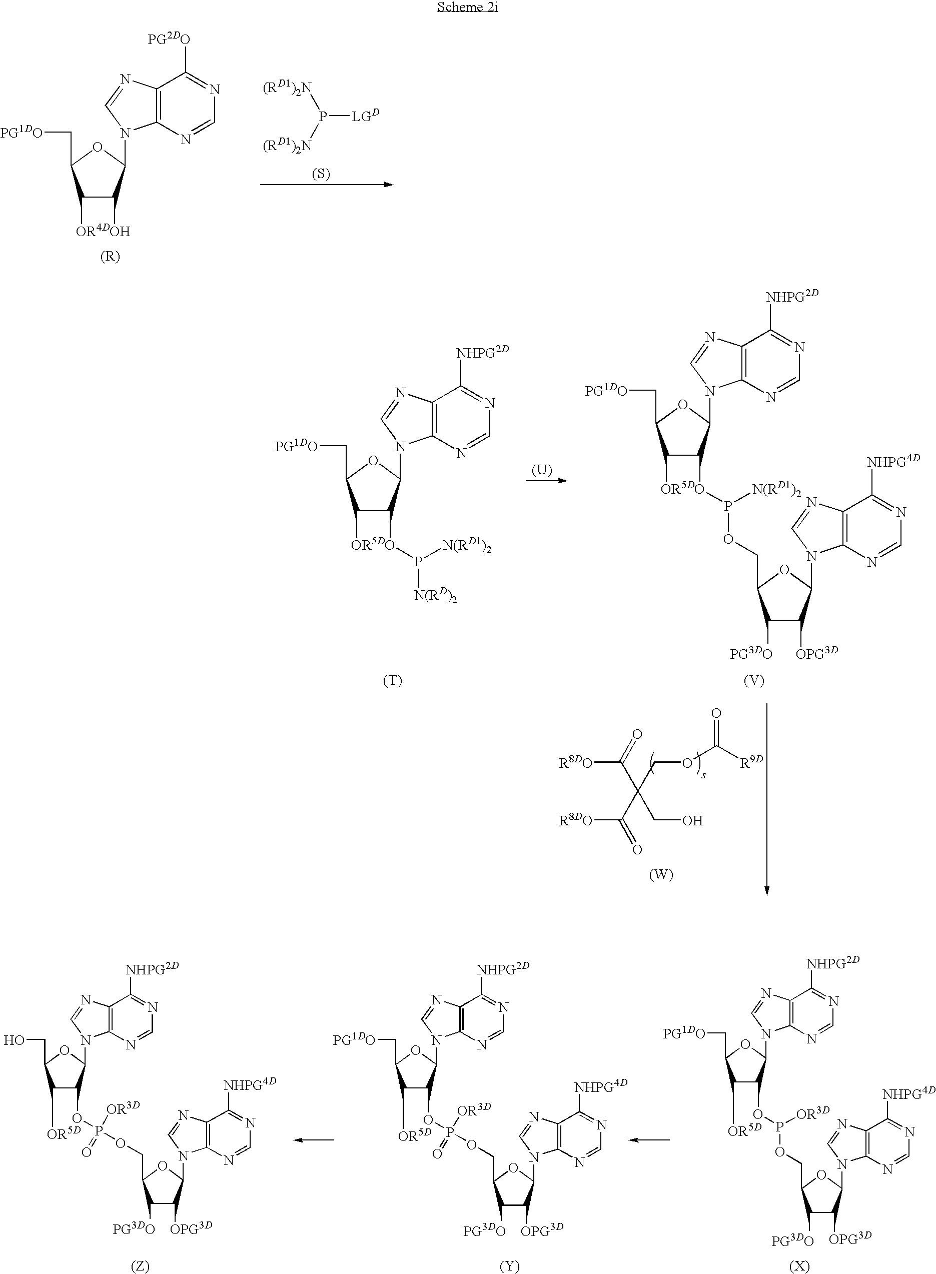

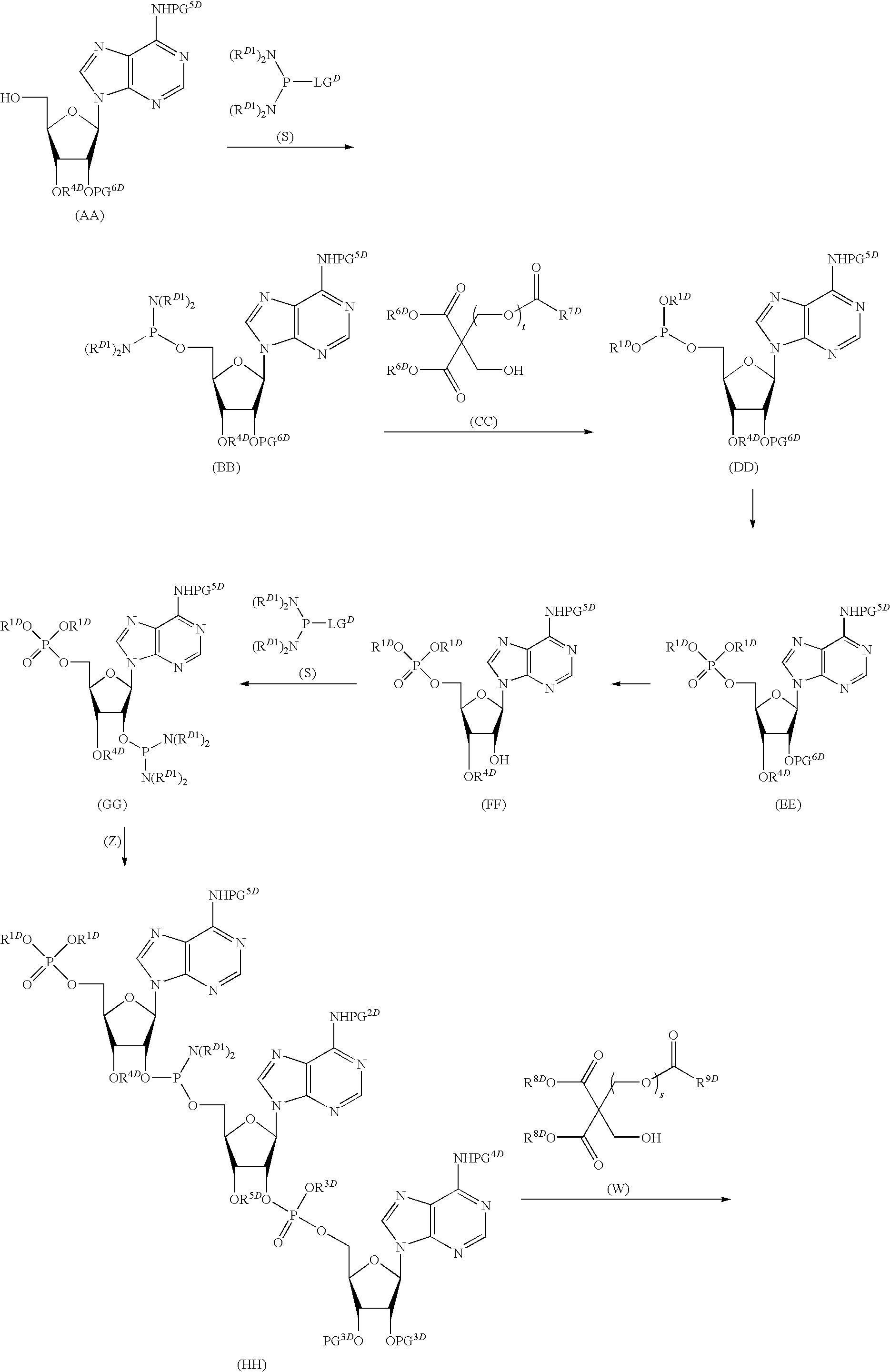

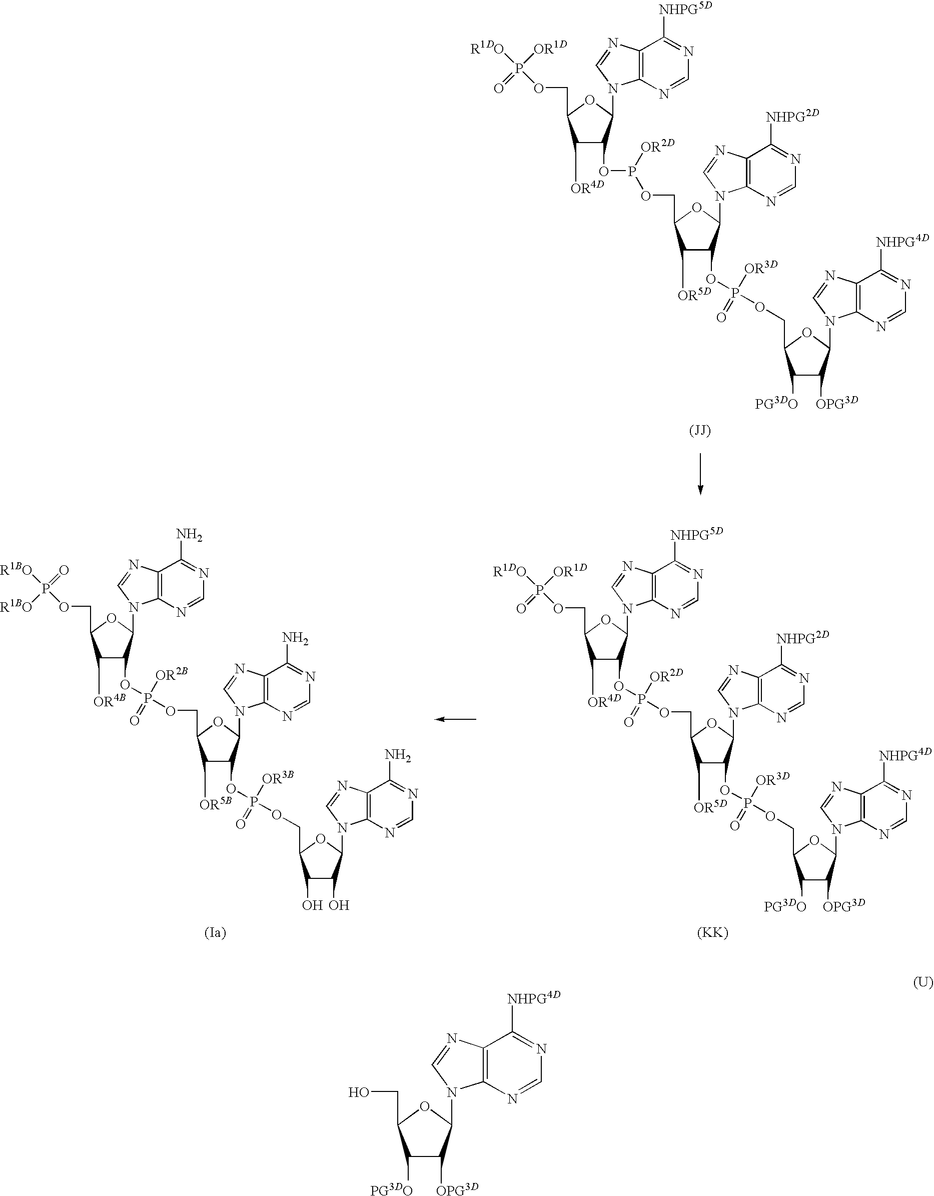

An embodiment described herein relates to a method of synthesizing a compound of Formula (Ia) as shown in Scheme 2i. In Scheme 2i, R^{1D}, R^{2D}, R^{3D}, R^{4D}, R^{5D}, R^{6D}, R^{7D}, R^{8D}, R^{9D}, t and s can be the same as R^{1B}, R^{2B}, R^{3B}, R^{4B}, R^{5B}, R^{6B}, R^{7B}, R^{8B}, R^{9B}, o and p, respectively, as described above with respect Formula (Ia). PG^{1D}, PG^{2D}, PG^{3D}, PG^{4D}, PG^{5D} and PG^{6D} represent appropriate protecting groups.

A phosphoamidite can be formed at the 2′-position of a compound of Formula R by reacting a compound of Formula S with the —OH attached to the 2′-position of a compound of Formula R to form a compound of Formula T. In an embodiment, each R^{D1} can be independently an optionally substituted C₁₋₄ alkyl, and LG^{D} can be a suitable leaving group. In some embodiments, the leaving group on a compound of Formula S can be a halogen.

A protected adenosine of Formula U can be added to a compound of Formula T to form a compound of Formula V. As shown in Scheme 2i, the —OH attached to the 5′-position on a compound of Formula U can be added to the phosphoamidite on a compound of Formula T. A R^{3D} moiety can be added to a compound of Formula V by reacting a compound of Formula V with a compound of Formula W to form a compound of Formula X. A compound of Formula W can be added to the phosphorous of a compound of Formula V to form a compound of Formula X. In one or both steps, an activator can be used to promote the reaction. One suitable class of activators is tetrazoles. Additional activators are described herein.

The phosphite of a compound of Formula X can be oxidized to a phosphate. In embodiment, the oxidation can be achieved using iodine and water. The protecting group, PG^{1D}, can be removed using methods known to those skilled in the art to form a compound of Formula Z. In some embodiments, PG^{1D} can be selectively removed, for example, PG^{1D} can be removed without removing one or more of the group of PG^{2D}, PG^{3D}, and PG^{4D}. In an embodiment, PG^{1D}, PG^{2D}, PG^{3D} and PG^{4D} can be chosen such that the conditions for removing PG^{1D} cannot remove PG^{2D}, PG^{3D} or PG^{4D}.

A phosphoamidite can be formed at the 5′-position of a compound of Formula AA by reacting a compound of Formula S with a compound of Formula AA to form a compound of Formula BB. In an embodiment, each R^{D1} can be independently an optionally substituted C₁₋₄ alkyl, and LG^{D} can be a suitable leaving group. In some embodiments, the leaving group on a compound of Formula S can be a halogen.

A R^{1D} moiety can be added to a compound of Formula BB by reacting a compound of Formula CC to a compound of Formula BB to form a compound of Formula DD. As shown above, the R^{1D} moiety can be added to the phosphorous on a compound of Formula BB. As described previously, an activator such as a tetrazole can be used to assist the addition of a compound of Formula CC to a compound of Formula BB.

The phosphite of a compound of Formula DD can be oxidized to a phosphate using an appropriate oxidizing agent and oxygen donor. In one embodiment, the oxidizing agent can be iodine and the oxygen donor can be water.

The protecting group, PG^{6D}, can be removed from a compound of Formula EE using methods known to those skilled in the art to form a compound of Formula FF. In an embodiment, PG^{6D} can be selectively removed. For example, PG^{6D} can be removed without removing PG^{5D}. In some embodiments, PG^{6D} can be a levulinoyl group. In other embodiments, PG^{6D} can be a silyl ether group. To remove PG^{6D} when PG^{6D} is a levulinoyl group, in an embodiment, a compound of Formula EE can be treated with hydrazinium acetate.

A phosphoamidite can be formed at the 2′-position of a compound of Formula FF by reacting a compound of Formula S with a compound of Formula FF to form a compound of Formula GG. In an embodiment, each R^{D1} can be independently an optionally substituted C₁₋₄ alkyl, and LG^{D} can be a suitable leaving group. In some embodiments, the leaving group on a compound of Formula S can be a halogen.

A compound of Formula Z can then be added to a compound of Formula GG to form a compound of Formula HH. As shown above, the —OH attached to the 5′-position of a compound of Formula Z can be added to the phosphoamidite of a compound of Formula GG to form a compound of Formula HH. A R^{2D} moiety can be added to a compound of Formula HH by reacting a compound of Formula W with a compound of Formula HH to form a compound of Formula JJ. As shown in Scheme 2i, a compound of Formula W can be added to the phosphorous of the phosphoadmidite of a compound of Formula HH to form a compound of Formula JJ. The addition of the compounds of Formulae Z and W to the compounds of Formulae GG and HH, respectively, can be facilitated by an activator such as a tetrazole.

The phosphite of a compound of Formula JJ can be oxidized to a phosphate to form a compound of Formula KK. In an embodiment, the oxidation can be accomplished using an oxidizing agent such as iodine and the oxygen donor such as water.

The protecting group moieties, PG^{2D} PG^{3D} PG^{4D} and PG^{5D} can be removed using conditions known to those skilled in the art to form a compound of Formula (Ia). In some embodiments, PG^{1D} can be a silyl ether. Examples of silyl ethers are described herein. In an embodiment, PG^{1D} can be removed with a tetra(alkyl)ammonium halide (e.g., tetra(t-butyl)ammonium fluoride (TBAF)). In some embodiments, one, two or all of the protecting groups represented by PG^{2D}, PG^{4D} and PG^{5D} can be a triarylmethyl protecting group. Suitable triarylmethyl protecting groups are described above. In an embodiment, PG^{2D}, PG^{4D} and PG^{5D} can be removed with an acid such as acetic acid or a zinc dihalide such as ZnBr₂. In some embodiments, each PG^{3D} can be a levulinoyl group. In other embodiments, each PG^{3D} can be a silyl ether group which can be removed using an appropriate reagent such as a tetraalkylammonium fluoride. If one or both of PG^{3C} are levulinoyl groups, the levulinoyl group(s) can be removed with hydrazinium acetate.

In some embodiments PG^{3D} can be selectively removed. In an embodiment, PG^{3D} can be removed without removing one or more selected from PG^{2D}, PG^{4D} and PG^{5D}. In other embodiments, one of more of PG^{2D}, PG^{4D} and PG^{5D} can be removed selectively. As an example, PG^{2D}, PG^{4D} and PG^{5D} can be chosen such that conditions that remove PG^{2D}, PG^{4D} and PG^{5D} cannot remove PG³. In an embodiment, PG^{3D} can be removed before removing one or more selected from PG^{2D}, PG^{4D} and PG^{5D}. In another embodiment, PG^{3D} can be removed after removing one or more selected from PG^{2D}, PG^{4D} and PG^{5D}. In an embodiment, PG^{2D}, PG^{4D} and PG^{5D} can be sequentially or substantially simultaneously.

Various protecting groups can be present on the compounds shown in Schemes 2i. One benefit of having these protecting groups is that the addition of one or more compounds can be directed to certain positions of another compound(s). Furthermore, as previously discussed, the protecting groups can block undesirable side reactions that may occur during later synthetic transformations. Minimization of unwanted side compound can make in the separation and isolation of the desired compound(s) more facile.

In Scheme 2i, the compounds of Formula S used form the phosphoamidites can be the same or different. Similarly, the compounds of Formula W in Schemes 21 can be the same or different.

The methods of synthesis described above in Schemes 2a, 2b, 2c, 2d, 2e, 2f, 2g, 2h and 2i can be used to synthesize any of the compounds and any embodiments described herein such as those of Formulae (I) and/or (Ia).

### Pharmaceutical Compositions

An embodiment described herein relates to a pharmaceutical composition, that can include a therapeutically effective amount of one or more compounds described herein (e.g., a compound of Formula (I) and/or a compound of Formula (Ia)) and a pharmaceutically acceptable carrier, diluent, excipient or combination thereof.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Suitable routes of administration may, for example, include oral, rectal, topical transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### Methods of Use

One embodiment disclosed herein relates to a method of treating and/or ameliorating a disease or condition that can include administering to a subject a therapeutically effective amount of one or more compounds described herein, such as a compound of Formula (I) and/or a compound of Formula (Ia), or a pharmaceutical composition that includes a compound described herein.

Some embodiments disclosed herein relate to a method of ameliorating or treating a neoplastic disease that can include administering to a subject suffering from a neoplastic disease a therapeutically effective amount of one or more compounds described herein (e.g., a compound of Formula (I) and/or a compound of Formula (Ia)) or a pharmaceutical composition that includes one or more compounds described herein. In an embodiment, the neoplastic disease can be cancer. In some embodiments, the neoplastic disease can be a tumor such as a solid tumor. In an embodiment, the neoplastic disease can be leukemia. Exemplary leukemias include, but are not limited to, acute lymphoblastic leukemia (ALL), acute myeloid leukemia (AML) and juvenile myelomonocytic leukemia (JMML).

An embodiment disclosed herein relates to a method of inhibiting the growth of a tumor that can include administering to a subject having a tumor a therapeutically effective amount of one or more compounds described herein or a pharmaceutical composition that includes one or more compounds described herein.

Other embodiments disclosed herein relates to a method of ameliorating or treating a viral infection that can include administering to a subject suffering from a viral infection a therapeutically effective amount of one or more compounds described herein or a pharmaceutical composition that includes one or more compounds described herein. In an embodiment, the viral infection can be caused by a virus selected from an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and/or a Togaviridae. In an embodiment, the viral infection can be a hepatitis C viral infection.

One embodiment disclosed herein relates to a method of ameliorating or treating a parasitic disease that can include administering to a subject suffering from a parasitic disease a therapeutically effective amount of one or more compounds described herein or a pharmaceutical composition that includes one or more compounds described herein. In an embodiment, the parasite disease can be Chagas' disease.

As used herein, a “subject” refers to an animal that is the object of treatment, observation or experiment. “Animal” includes cold- and warm-blooded vertebrates and invertebrates such as fish, shellfish, reptiles and, in particular, mammals.

“Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “therapeutically effective amount” is used to indicate an amount of an active compound, or pharmaceutical agent, that elicits the biological or medicinal response indicated. For example, a therapeutically effective amount of compound can be the amount need to prevent, alleviate or ameliorate symptoms of disease or prolong the survival of the subject being treated This response may occur in a tissue, system, animal or human and includes alleviation of the symptoms of the disease being treated. Determination of a therapeutically effective amount is well within the capability of those skilled in the art, especially in light of the detailed disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. (See e.g., Fingl et al. 1975, in “The Pharmacological Basis of Therapeutics”, which is hereby incorporated herein by reference in its entirety, with particular reference to Ch. 1, p. 1). The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the patient. In some embodiments, the compounds will be administered for a period of continuous therapy, for example for a week or more, or for months or years.

In instances where human dosages for compounds have been established for at least some condition, those same dosages my be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Alternatively dosages may be based and calculated upon the surface area of the patient, as understood by those of skill in the art.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

### EXAMPLES

Additional embodiments are disclosed in further detail in the following examples, which are not in any way intended to limit the scope of the claims.

### 2-ACETYL-2-(HYDROXYMETHYL)-3-OXOBUTYL ACETATE (1)

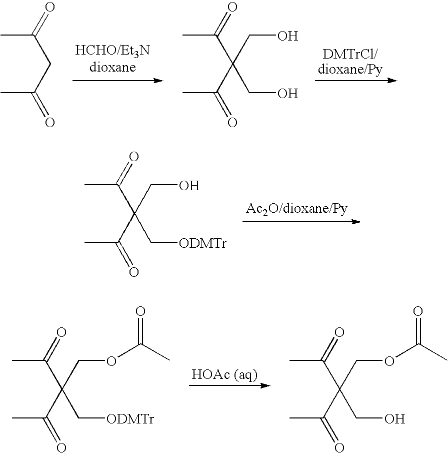

### 2-ACETYL-2-HYDROXYMETHYL-3-OXOBUTYL ACETATE (2)

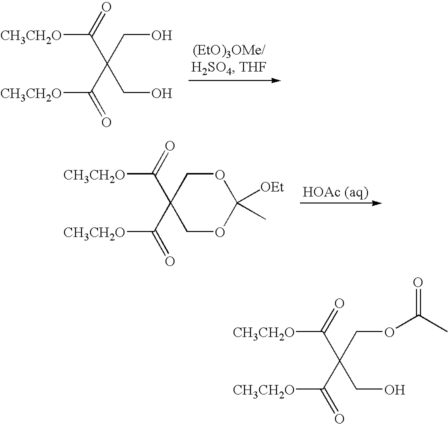

Diethyl 2-ethoxy-2-methyl-1,3-dioxane-5,5-dicarboxylate. Concentrated H₂SO₄ (1.3 mmol; 71 μL) was added to a mixture of diethyl 2,2-bis(hydroxymethyl)malonate (43.5 mmol, 9.6 g) and triethyl orthoacetate (65.2 mmol; 11.9 mL) in dry THF (15 mL). The reaction was allowed to proceed overnight and the mixture was the poured into an ice-cold solution of 5% NaHCO₃ (50 mL). The product was extracted with diethyl ether (2×50 mL), washed with saturated aqueous NaCl (2×50 mL) and dried over Na₂SO₄. The solvent was evaporated and the crude product was purified on a silica gel column eluting with a mixture of dichloromethane and methanol (95:5, v/v). The product was obtained as clear oil in 89% yield (11.3 g). ¹H NMR δ_{H} (500 MHz, CDCl₃): 4.30-4.36 (m, 6H, 4-CH₂, 6-CH₂ and 5-COOCH₂Me), 4.18 (q, J=7.1 Hz, 5-COOCH₂Me), 3.54 (q, J=7.10 Hz, 2H, 2-OCH₂Me), 1.46 (s, 3H, 2-CH₃), 1.32 (t, J=7.10 Hz, 3H, 2-OCH₂Me), 1.27 (t, J=7.1 Hz 3H, 5-COOCH₂Me), 1.26 (t, J=7.1 Hz 3H, 5-COOCH₂Me). ¹³C NMR (500 MHz, CDCl₃): δ=168.0 and 167.0 (5-COOEt), 111.1 (C2), 62.0 and 61.9 (5-COOCH₂Me), 61.6 (C4 and C6), 58.7 (2-OCH₂Me), 52.3 (C5), 22.5 (2-Me), 15.1 (2-OCH₂CH₃), 14.0 and 13.9 (5-COOCH₂CH₃).

Diethyl 2-(acetyloxymethyl)-2-(hydroxymethyl)malonate. Diethyl 2-ethoxy-2-methyl-1,3-dioxane-5,5-dicarboxylate (17.9 mmol; 5.2 g) was dissolved in 80% aqueous acetic acid (30 mL) and left for 2 h at room temperature. The solution was evaporated to dryness and the residue was coevaporated three times with water. The product was purified by silica gel column chromatography eluting with ethyl acetate in dichloromethane (8:92, v/v). The product was obtained as yellowish oil in 75% yield (3.6 g). ¹H NMR δ_{H} (500 MHz, CDCl₃): 4.76 (s, 2H, CH₂OAc), 4.26 (q, J=7.10 Hz, 4H, OCH₂Me), 4.05 (d, J=7.10 Hz, 2H, CH₂OH), 2.72 (t, J=7.1 Hz, 1H, CH₂OH), 2.08 (s, 3H, Ac), 1.27 (t, J=7.10 Hz, 6H, OCH₂CH₃). ¹³C NMR (500 MHz, CDCl₃): δ=170.9 (C═O Ac), 168.1 (2×C═O malonate), 62.3 and 62.2 (CH₂OH and CH₂OAc), 61.9 (2×OCH₂CH₃) δ9.6 (spiro C), 20.7 (CH₃ Ac), 14.0 (2×OCH₂CH₃).

### 2-ACETYL-2-(HYDROXYMETHYL)-3-OXOBUTYL PIVALATE (3)

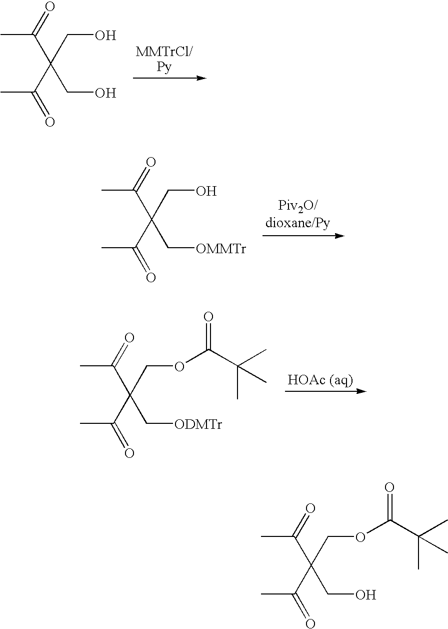

### 2,2-BIS(ETHOXYCARBONYL)-3-HYDROXYPROPYL PIVALATE (4)

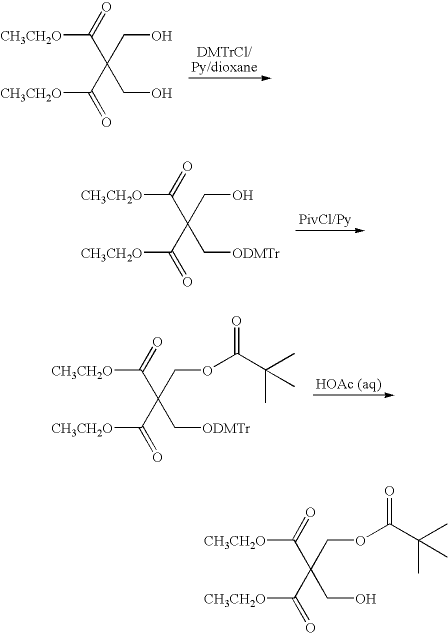

2,2-Bis(ethoxycarbonyl)-3-(4,4′-dimethoxytrityloxy)propyl pivalate. Diethyl 2,2-bis(hydroxymethyl)malonate was reacted with 1 equiv. of 4,4′-dimethoxytrityl chloride in 1,4-dioxane containing 1 equiv. of pyridine. Diethyl 2-(4,4′-dimethoxytrityloxymethyl)-2-(hydroxymethyl)malonate obtained (2.35 g, 4.50 mmol) was acylated with pivaloyl chloride (0.83 mL, 6.75 mmol) in dry MeCN (10 mL) containing 3 equiv. pyridine (1.09 mL, 13.5 mmol). After 3 days at room temperature, the reaction was quenched with MeOH (20 mL) and a conventional CH₂Cl₂/aq HCO₃⁻— workup was carried out. Silica gel chromatography (EtOAc/hexane 1:1, v/v) gave 2.47 g (90%) of the desired product as yellowish syrup. ¹H NMR (CDCl₃, 200 MHz): 7.13-7.39 [m, 9H, (MeO)₂ Tr]; 6.81 (d, 4H, [MeO]₂ Tr); 4.71 (s, 2H, CH₂OPiv); 4.15 (q, J=7.1, 4H, OCH₂CH₃); 3.78 [s, 6H, (CH₃O)₂Tr]; 3.67 (s, 2H, CH₂ODMTr); 1.27 (t, J=7.1, 6H, OCH₂CH₃); 1.02 [s, 9H, COC(CH₃)₃].

2,2-Bis(ethoxycarbonyl)-3-hydroxypropyl pivalate. 2,2-Bis(ethoxycarbonyl)-3-(4,4′-dimethoxytrityloxy)propyl pivalate (2.47 g, 4.07 mmol) in a 4:1 mixture of CH₂Cl₂ and MeOH (20 mL) was treated for 4 hours at room temperature with TFA (2.00 mL, 26.0 mmol) to remove the dimethoxytrityl group. The mixture was neutralized with pyridine (2.30 mL, 28.6 mmol), subjected to CH₂Cl₂/aq workup and purified by Silica gel chromatography (EtOAc/hexane 3:7, v/v) to obtain 1.15 g (93%) of the desired product. ¹H NMR (CDCl₃, 200 MHz): 4.59 (s, 2H, CH₂OPiv); 4.25 (q, J=7.1, 4H, OCH₂CH₃); 4.01 (s, 2H, CH₂OH); 1.28 (t, J=7.1, 6H, OCH₂CH₃); 1.18 [s, 9H, COC(CH₃)₃]. ESI−MS⁺: m/z 305.4 ([MH]⁺), 322.6 ([MNH₄]⁺), 327.6 ([MNa]⁺), 343.5 ([MK]⁺).

### DIETHYL 2-ACETYLOXYMETHYL-2-HYDROXYMETHYLMALONATE (5)

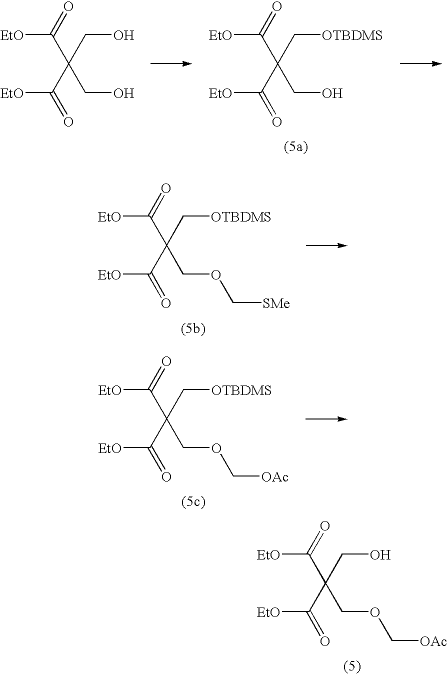

Diethyl 2-(tert-butyldimethylsilyloxymethyl)-2-hydroxymethylmalonate (5a). Diethyl 2,2-bis(hydroxymethyl)malonate (28.3 mmol; 6.23 g) was coevaporated twice from dry pyridine and dissolved in the same solvent (20 mL). tert-Butyldimethylsilyl chloride (25.5 mmol; 3.85 g) in dry pyridine (10 mL) was added portionwise. The reaction was allowed to proceed for 4 days. The mixture was evaporated to a solid foam, which was then equilibrated between water (200 mL) and DCM (4×100 mL). The organic phase was dried on Na₂SO₄. The product was purified by silica gel chromatography eluting with 10% ethyl acetate in DCM. The yield was 78%. ¹H NMR (CDCl₃) δ 4.18-4.25 (m, 4H, OCH₂Me), 4.10 (s, 2H, CH₂OSi), 4.06 (s, 2H, CH₂OH), 2.63 (br s, 1H, OH), 1.26 (t, J=7.0 Hz, 6H, OCH₂CH₃), 0.85 (s, 9H, Si-SMe₃), 0.05 (s, 6H, Me-Si). ¹³C NMR (CDCl₃) δ 169.2 (C═O), 63.3 (CH₂OH), 62.8 (CH₂OSi), 61.6 (spiro C), 61.4 (OCH₂Me), 25.6 [C(CH₃)₃], 18.0 (Si-CMe₃), 14.0 (OCH₂CH₃), −3.6 (Si—CH₃). MS [M+H]+obsd. 335.7, calcd. 335.2; [M+Na] obsd. 357.6, calcd. 357.2.

Diethyl 2-(tert-butyldimethylsilyloxymethyl)-2-methylthiomethylmalonate (5b). Compound 5a (19.7 mmol; 6.59 g) was dissolved into a mixture of acetic anhydride (40 mL), acetic acid (12.5 mL) and DMSO (61 mL) and the mixture was stirred overnight. The reaction was stopped by dilution with cold aq Na₂CO₃ (290 ml 10% aq solution) and the product was extracted in diethyl ether (4×120 mL). The combined organic phase was dried on Na₂SO₄. The product was purified by silica gel chromatography using DCM as an eluent. The yield was 91%. ¹H NMR (CDCl₃) δ 4.61 (s, 2H, OCH₂S), 4.14-4.19 (m, 4H, OCH₂Me), 4.06 (s, 2H, CH₂OSi), 4.00 (s, 2H, CH₂OCH₂SMe), 2.06 (SCH₃), 1.22 (t, J=7.0 Hz, 6H, OCH₂CH₃), 0.83 (s, 9H, Si-SMe₃), 0.02 (s, 6H, Me-Si). ¹³C NMR (CDCl₃) δ 168.3 (C═O), 75.6 (CH₂S), 65.7 (CH₂OCH₂SMe), 61.4 (CH₂OSi), 61.2 (spiro C), 60.9 (OCH₂Me), 25.6 [C(CH₃)₃], 18.0 (Si—CMe₃), 14.0 (OCH₂CH₃), 13.7 (SCH₃), −3.6 (S₁—CH₃). MS [M+H]⁺ obsd. 395.4, calcd. 395.2; [M+Na]⁺ obsd. 417.6, calcd. 417.2.

Diethyl 2-acetyloxymethyl-2-(tert-butyldimethylsilyloxymethyl)malonate (5c). Compound 5b (17.9 mmol; 7.08 g) was dissolved in dry DCM (96 mL) under nitrogen. Sulfurylchloride (21.5 mmol; 1.74 mL of 1.0 mol L⁻¹ solution in DCM) was added in three portions and the mixture was stirred for 70 min under nitrogen. The solvent was removed under reduced pressure and the residue was dissolved into dry DCM (53 mL). Potassium acetate (30.9 mmol; 3.03 g) and dibenzo-18-crown-6 (13.5 mmol; 4.85 g) in DCM (50 mL) were added and the mixture was stirred for one hour and a half. Ethyl acetate (140 mL) was added, the organic phase was washed with water (2×190 mL) and dried on Na₂SO₄. The product was purified by silica gel chromatography using DCM as an eluent. The yield was 71%. ¹H NMR (CDCl₃) δ 5.24 (s, 2H, OCH₂O), 4.15-4.22 (m, 4H, OCH₂Me), 4.13 (s, 2H, CH₂OSi), 4.08 (s, 2H, CH₂OAc), 2.08 (Ac), 1.26 (t, J=8.0 Hz, 6H, OCH₂CH₃), 0.85 (s, 9H, Si-SMe₃), 0.04 (s, 6H, Me-Si). ¹³C NMR (CDCl₃) δ 170.2 (Ac), 168.0 (C═O), 89.3 (OCH₂O), 67.5 (CH₂OAc), 61.4 (OCH₂Me), 61.1 (CH₂OSi), 60.2 (spiro C), 25.6 [C(CH₃)₃], 21.0 (Ac), 18.1 (Si—CMe₃), 14.0 (OCH₂CH₃), −5.7 (S1-CH₃). MS [M+Na]⁺ obsd. 429.6, calcd. 429.2.

Diethyl 2-acetyloxymethyl-2-hydroxymethylmalonate (5). Compound 5c (7.2 mmol; 2.93 g) was dissolved in dry THF (23 mL) and trietylamine trihydrogenfluoride (8.64 mmol; 1.42 mL) was added. The mixture was stirred for one week. Aq triethylammonium acetate (13 mL of 2.0 mol L⁻¹ solution) was added. The mixture was evaporated to dryness and the residue was purified by silica gel chromatography using DCM containing 2-5% MeOH as an eluent. The yield was 74%. ¹H NMR (CDCl₃) δ 5.25 (s, 2H, OCH₂O), 4.16-4.29 (m, 6H, OCH₂Me and CH₂OAc), 4.13 (s, 2H, CH₂OH), 2.10 (Ac), 1.81 (br s, 1H, OH), 1.26 (t, J=9.0 Hz, 6H, OCH₂CH₃). MS [M+Na]⁺ obsd. 315.3, calcd. 315.1.

### (2′,3′-O-LEV)-N⁶-(4-METHOXYTRITYL)-ADENOSINE (6)

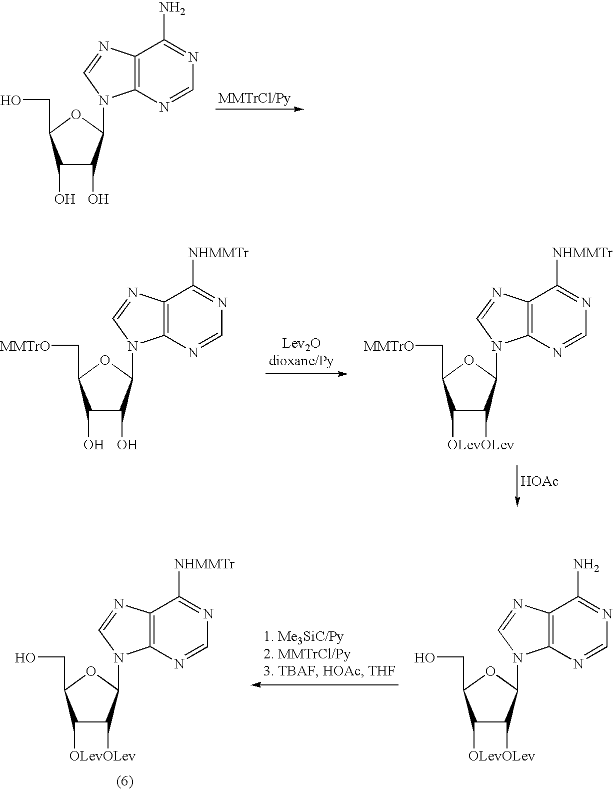

### (3′-O-PIVOXYMETHYL)-(5′-O-TBDMSO)-N⁶-(4-METHOXYTRITYL)-ADENOSINE

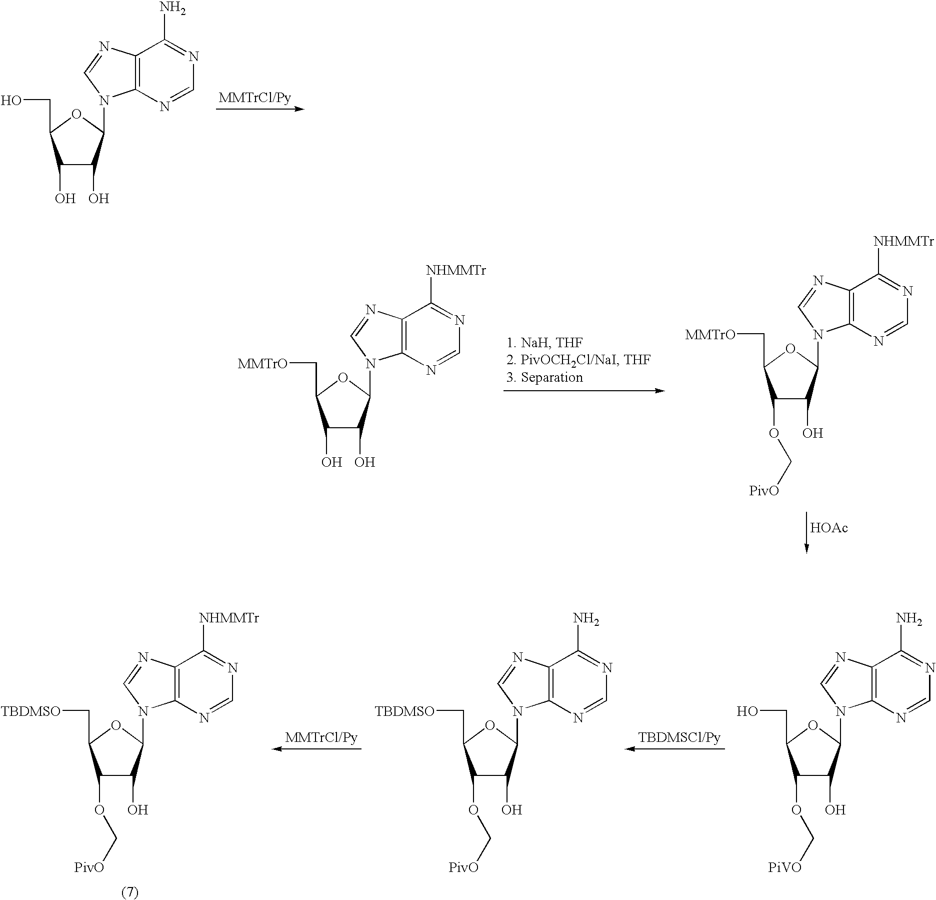

### (2′-O-LEV)-(3′-O-METHYL)-N′-(4-METHOXYTRITYL)-ADENOSINE (8)

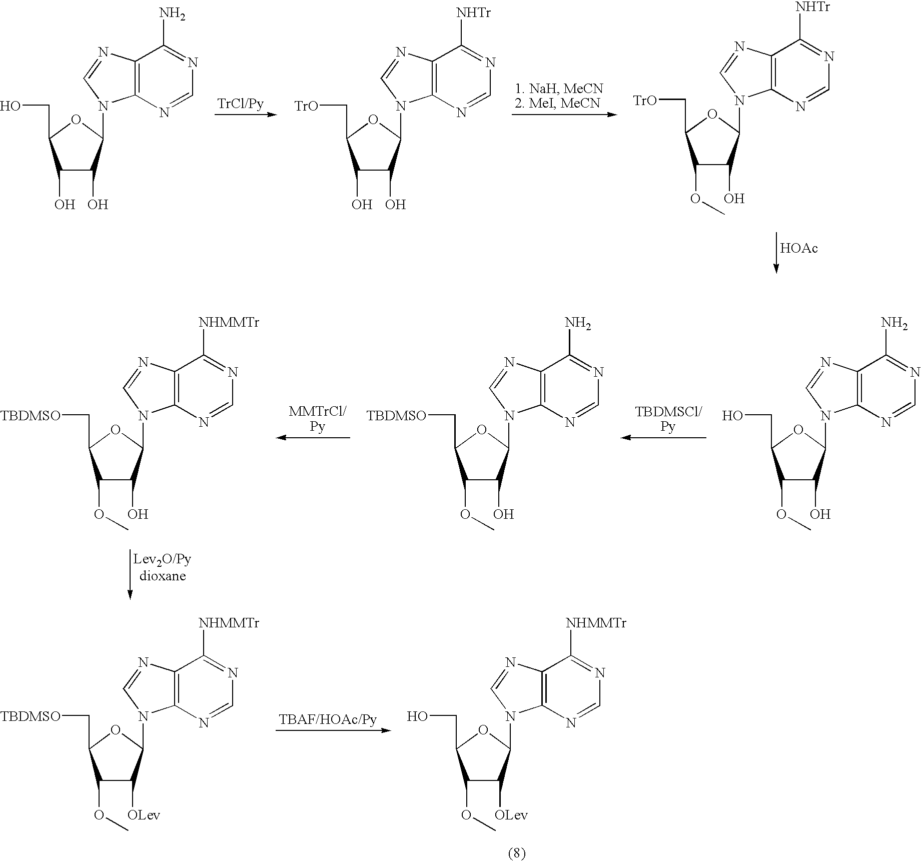

### (3′-O-ACETYLOXYMETHYL)-(2′-O-LEV)-N′-(4-METHOXYTRITYL)-ADENOSINE (9)

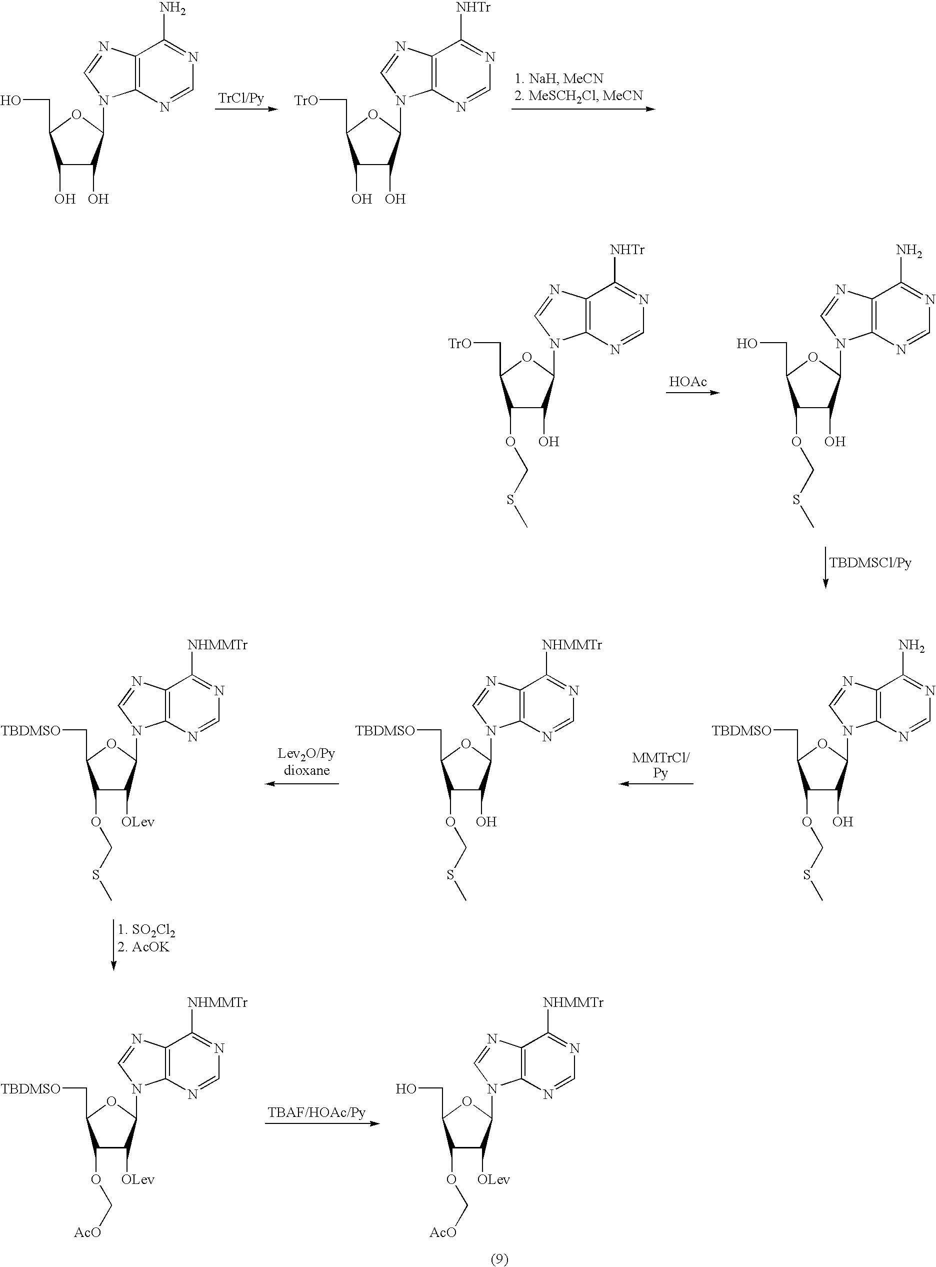

### (3′-O-ACETYLOXYMETHYL)-N⁶-(4-METHOXYTRITYL)-ADENOSINE (10)

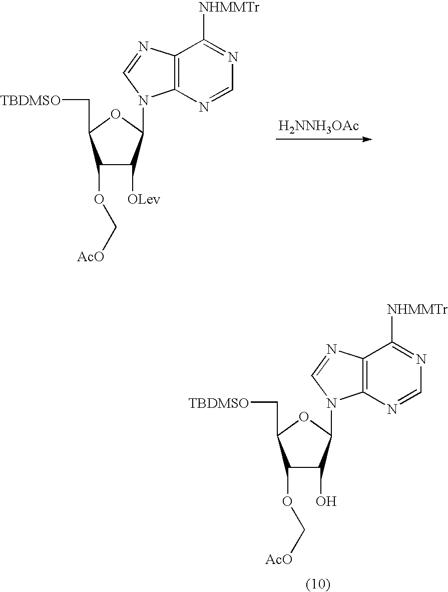

### TRIMERS (13), (14) (15) & (16)

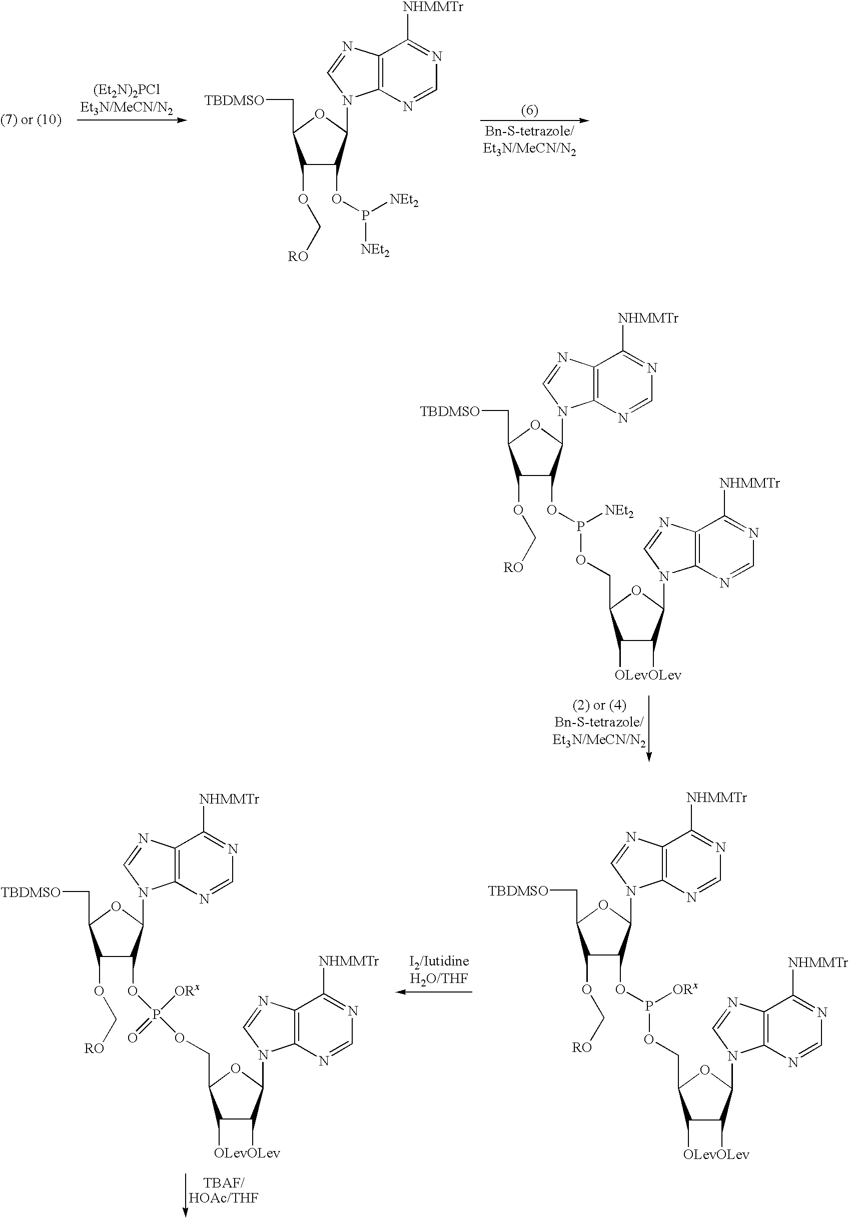

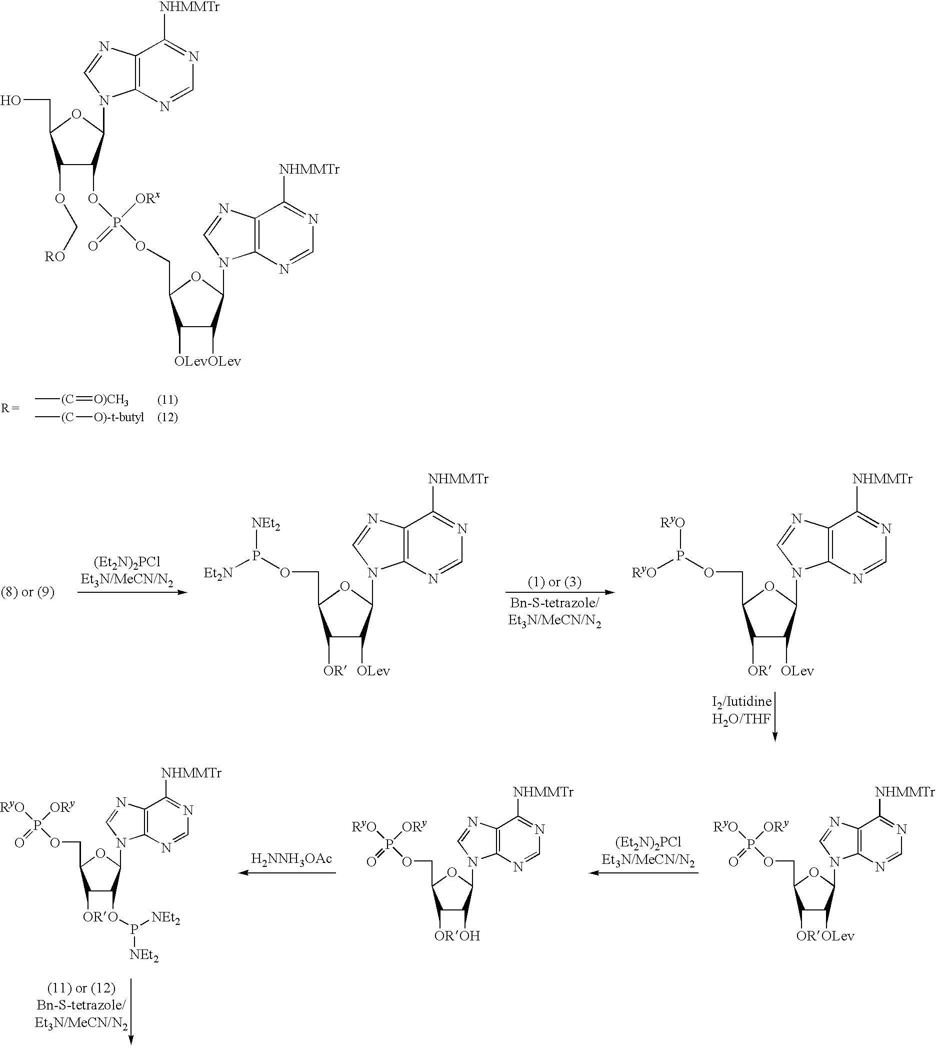

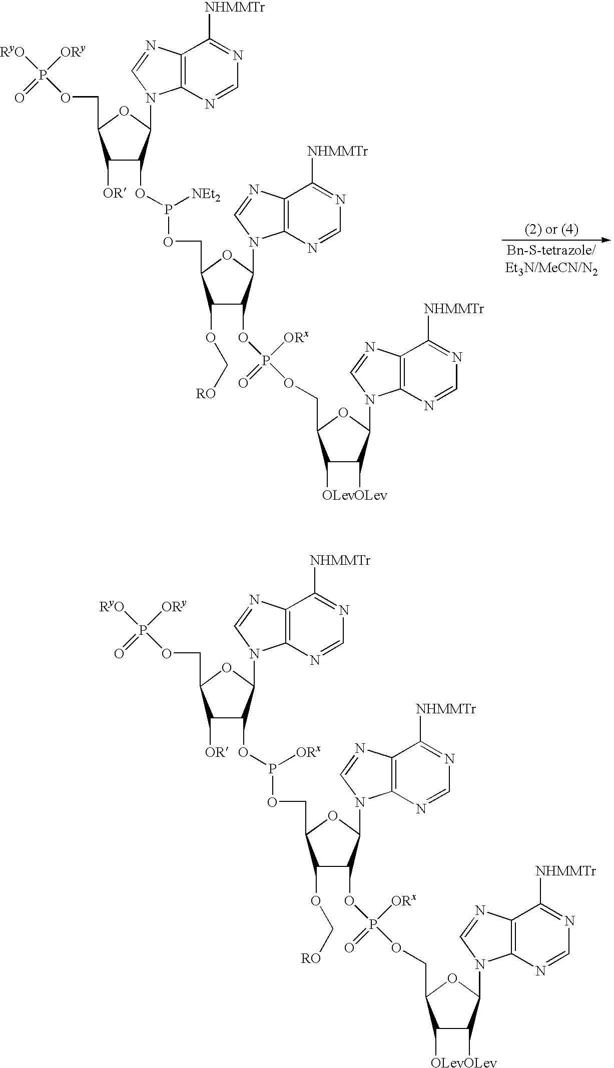

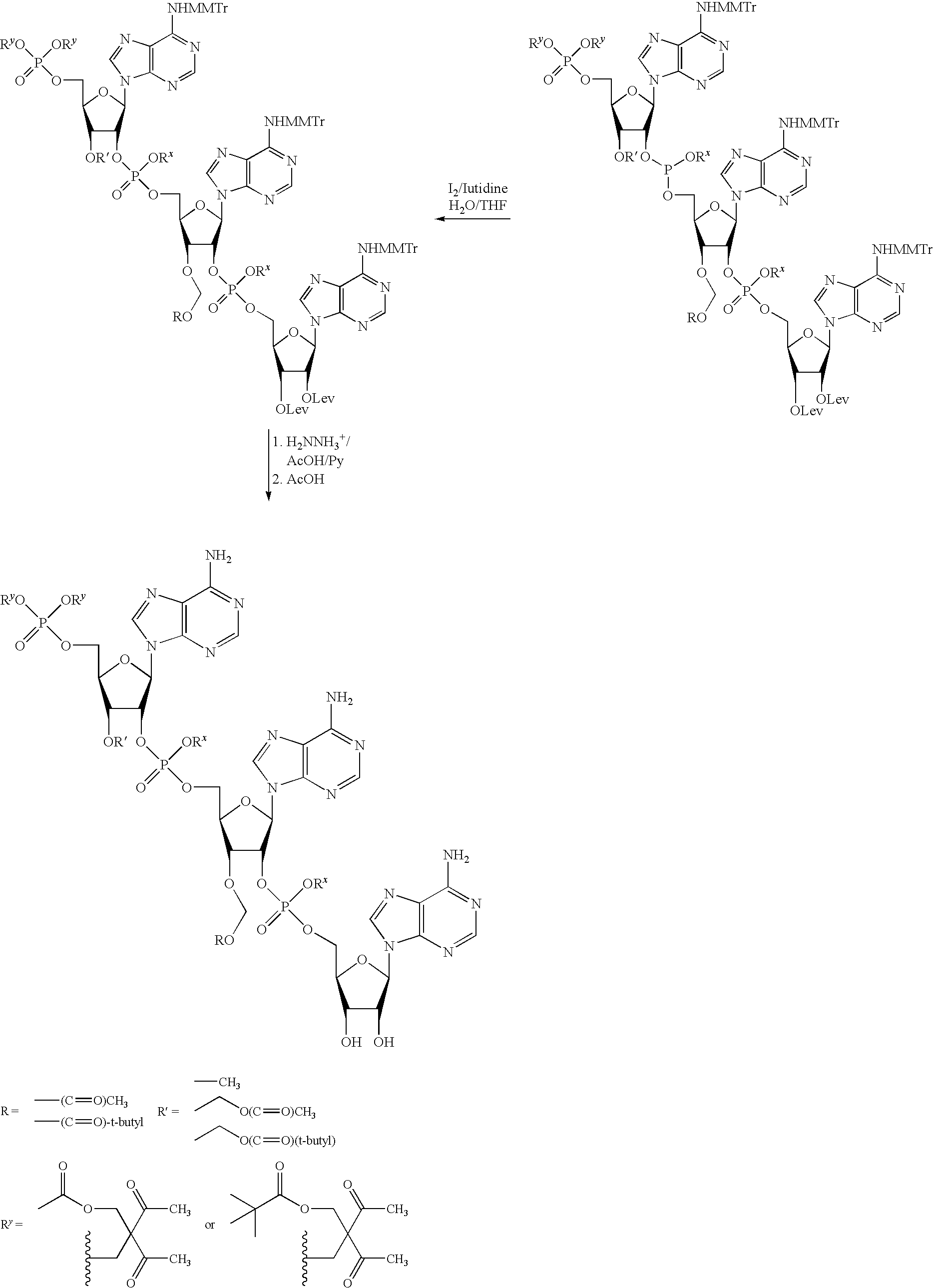

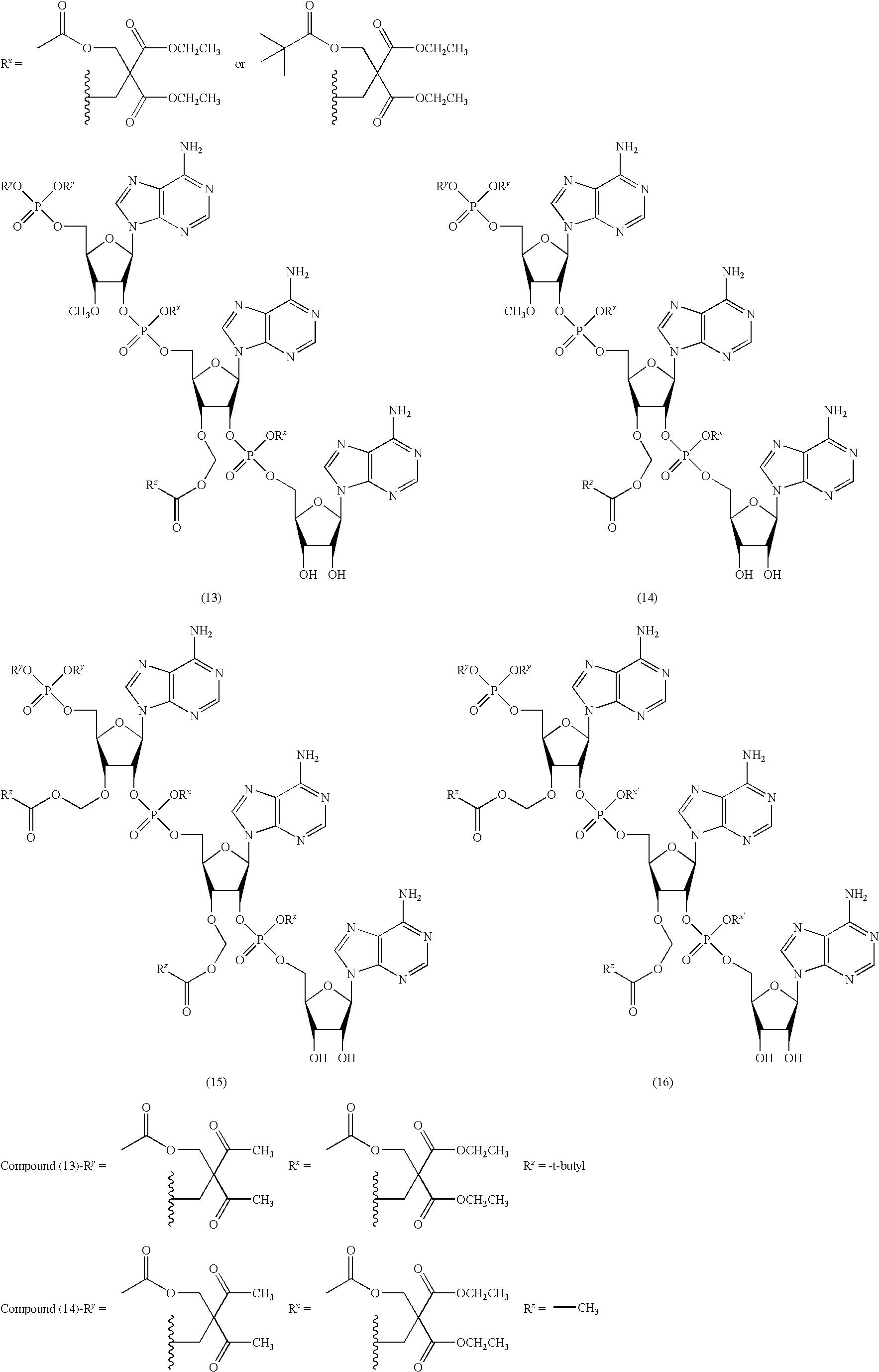

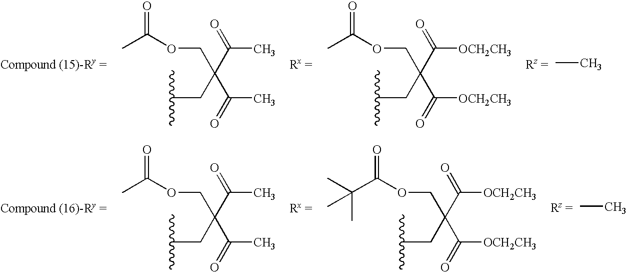

### Kinetic Studies

Preparation of the cell extract. 10×10⁶ of human prostate carcinoma cells (PC3) are treated with 10 mL of RIPA-buffer [15 mM Tris-HCl pH 7.5, 120 mM NaCl, 25 mM KCl, 2 mM EDTA, 2 mM EGTA, 0.1% Deoxycholic acid, 0.5% Triton X-100, 0.5% PMSF supplemented with Complete Protease Inhibitor Cocktail (Roche Diagnostics GmBH, Germany)] at 0° C. for 10 min. Most of the cells are disrupted by this hypotonic treatment and the remaining ones are disrupted mechanically. The cell extract obtained is centrifuged (900 rpm, 10 min) and the pellet is discarded. The extract is stored at −20° C.

Stability of Trimers (13), (14), (15) & (16) in the cell extract. The cell extract is prepared as described above (1 mL), and is diluted with a 9-fold volume of HEPES buffer (0.02 mol L⁻¹, pH 7.5, I=0.1 mol L⁻¹ with NaCl). A trimer (0.1 mg) is added into 3 mL of this HEPES buffered cell extract and the mixture is kept at 22±1° C. Aliquots of 150 μL are withdrawn at appropriate intervals, filtered with SPARTAN 13A (0.2 μm) and cooled in an ice bath. The aliquots are analyzed immediately by HPLC-ESI mass spectroscopy (Hypersil RP 18, 4.6×20 cm, 5 μm). For the first 10 min, 0.1% aq formic acid containing 4% MeCN is used for elution and then the MeCN content is increased to 50% by a linear gradient during 40 min.

Stability of Trimers (13), (14), (15) & (16) towards Porcine Liver Esterase. A trimer (1 mg) and 3 mg (48 units) of Sigma Porcine Liver Esterase (66H7075) are dissolved in 3 mL of HEPES buffer (0.02 mol L⁻¹, pH 7.5, I=0.1 mol L⁻¹ with NaCl). The stability test is carried out as described above for the cell extract.

Stability tests in human serum. Stability tests in human serum are carried out as described for the whole cell extract. The measurements are carried out in serum diluted 1:1 with HEPES buffer (0.02 mol L⁻¹, pH 7.5, I=0.1 mol L⁻¹ with NaCl).

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

What is claimed is:

1. A compound of Formula (I), or a pharmaceutically acceptable salt, prodrug or prodrug ester thereof: 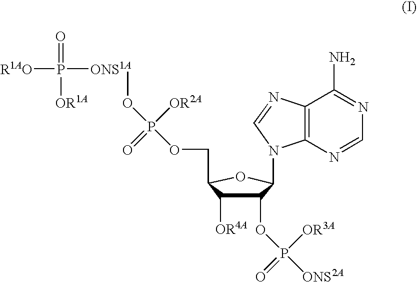
wherein:
each R^{1A} is 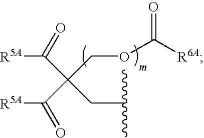
R^{2A} is 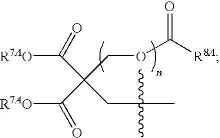
R^{3A} is 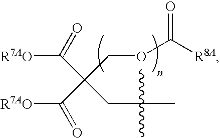
wherein R^{2A} and R^{3A} can be the same or different;
R^{4A} is —H or —C(R^{9A})₂—O—C(═O)R^{10A};
each R^{5A} each R^{6A}, each R^{7A}, each R^{8A}, each R^{9A} and R^{10A} are each independently hydrogen or an optionally substituted C₁₋₄-alkyl;
each m is independently 1 or 2;
each n is independently 1 or 2; and
NS^{1A} and NS^{2A} are independently selected from the group consisting of a nucleoside, a protected nucleoside, a nucleoside derivative and a protected nucleoside derivative.

2. The compound of claim 1, wherein each R^{5A} is an optionally substituted C₁₋₄ alkyl.

3. The compound of claim 1, wherein R^{6A} is an optionally substituted C₁₋₄ alkyl.

4. The compound of claim 1, wherein each R^{7A} is an optionally substituted C₁₋₄ alkyl.

5. The compound of claim 1, wherein R^{8A} is an optionally substituted C₁₋₄ alkyl.

6. The compound of claim 1, wherein both R^{9A} are hydrogen and R^{10A} is an optionally substituted C₁₋₄ alkyl.

7. The compound of claim 1, wherein each R^{1A}, R^{2A} and R^{3A} are each independently:

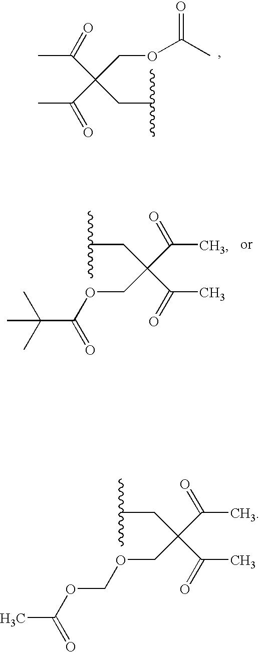

8. The compound of claim 1, wherein NS^{1A} has the structure: 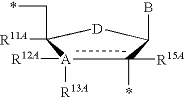
wherein: [symbol]
is single or double bond;
A is selected from the group consisting of C, O and S;
B is an optionally substituted heterocyclic base or a derivative thereof;
D is C═CH₂ or O;
R^{11A} is selected from the group consisting of hydrogen, azido, —CN, an optionally substituted C₁₋₄ alkyl and an optionally substituted C₁₋₄ alkoxy;
R^{12A} is absent or selected from the group consisting of hydrogen, halogen, hydroxy and an optionally substituted C₁₋₄ alkyl;
R^{13A} is absent or selected from the group consisting of hydrogen, halogen, azido, amino, hydroxy, an optionally substituted C₁₋₄ alkoxy and —OC(R^{16A})₂—O—C(═O)R^{17A};
R^{15A} is absent or selected from the group consisting of hydrogen, halogen, hydroxy, —CN, —NC, an optionally substituted C₁₋₄ alkyl, an optionally substituted haloalkyl and an optionally substituted hydroxyalkyl;
each R^{16A} and R^{17A} are independently hydrogen or an optionally substituted C₁₋₄-alkyl; and
* represents a point of attachment.

9. The compound of claim 7, wherein NS^{1A} is selected from the group consisting of: 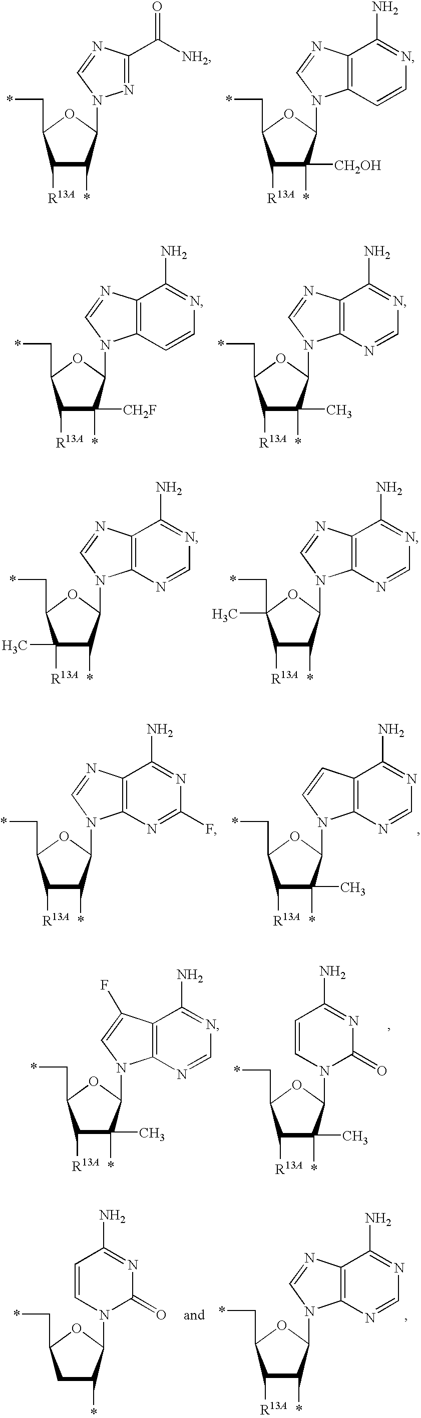
wherein:
* represents a point of attachment.

10. The compound of claim 1, wherein NS^{2A} has the structure: 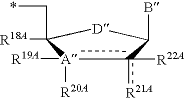
wherein: [symbol]
is single or double bond;
A″ is selected from the group consisting of C, O and S;
B″ is an optionally substituted heterocyclic base or a derivative thereof;
D″ is C═CH₂ or O;
R^{18A} is selected from the group consisting of hydrogen, azido, —CN, an optionally substituted C₁₋₄ alkyl and an optionally substituted C₁₋₄ alkoxy;
R^{19A} is absent or selected from the group consisting of hydrogen, halogen, hydroxy and an optionally substituted C₁₋₄ alkyl;
R^{20A} is absent or selected from the group consisting of hydrogen, halogen, azido, amino and hydroxy;
R^{21A} is selected from the group consisting of hydrogen, halogen, hydroxy, —CN, —NC, an optionally substituted C₁₋₄ alkyl and an optionally substituted C₁₋₄ alkoxy;
R^{22A} is absent or selected from the group consisting of hydrogen, halogen, hydroxy, —CN, —NC, an optionally substituted C₁₋₄ alkyl, an optionally substituted haloalkyl and an optionally substituted hydroxyalkyl, or when the bond to R^{21A} indicated by [symbol] is a double bond, then R^{21A} is a C₁₋₄ alkenyl and R^{22A} is absent; and
* represents a point of attachment.

11. The compound of claim 10, wherein NS^{2A} is selected from the group consisting of: 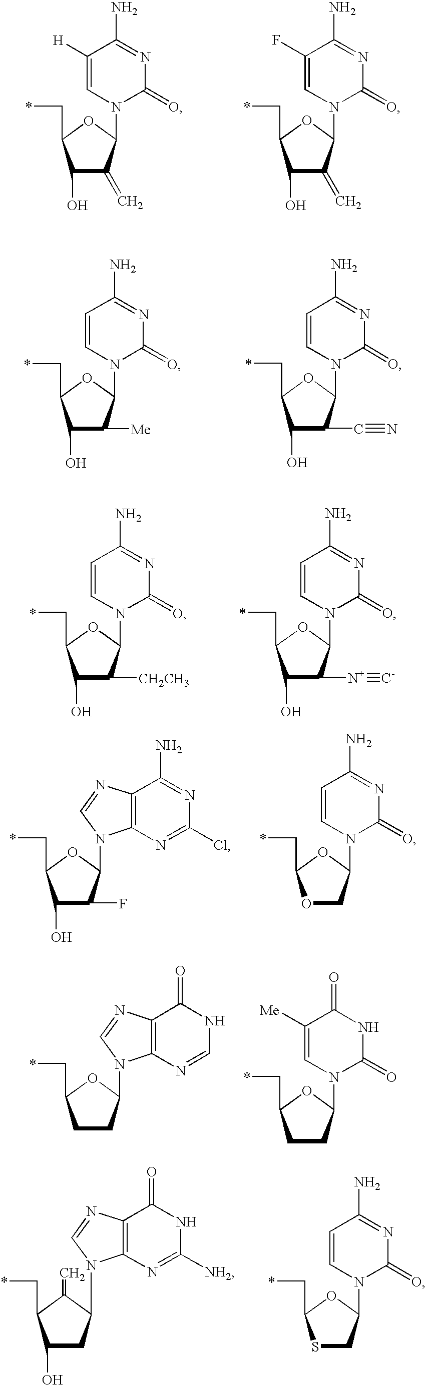 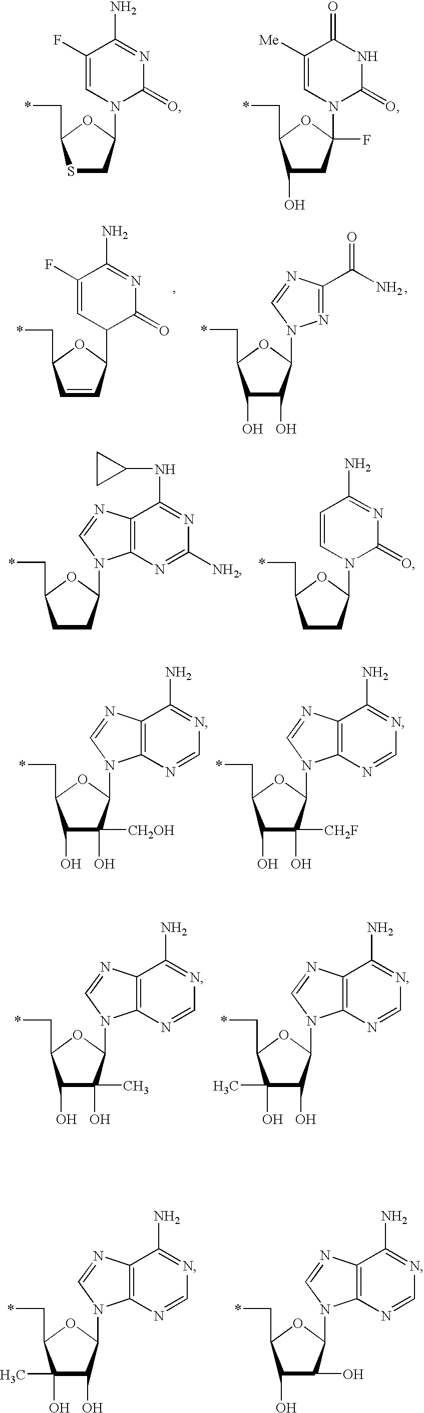 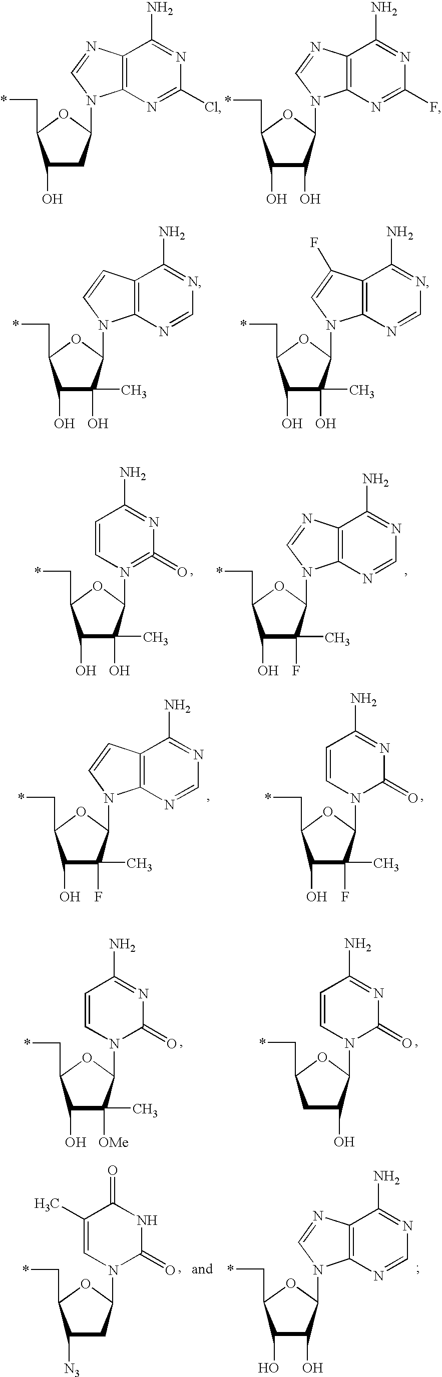
wherein * represents a point of attachment.

12. The compound of claim 1, wherein:
NS^{1A} is 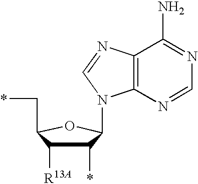
and NS^{2A} is 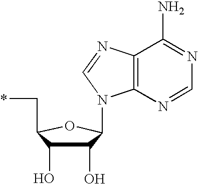
wherein:
R^{13A} is selected from the group consisting of —OH, an optionally substituted C₁₋₄ alkoxy and —OC(R^{16A})₂—O—C(═O)R^{17A};
each R^{16A} and R^{17A} are independently hydrogen or an optionally substituted C₁₋₄-alkyl; and
* represents a point of attachment.

13. A compound of Formula (Ia), or a pharmaceutically acceptable salt, prodrug or prodrug ester thereof: 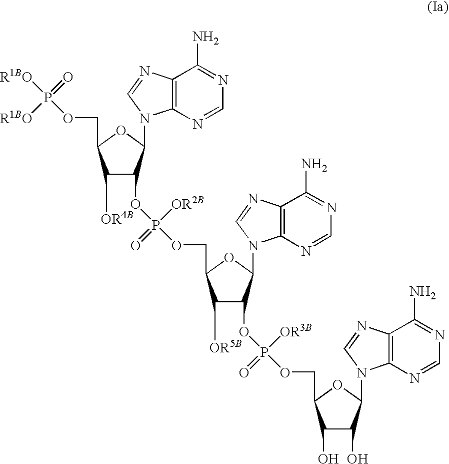
wherein:
each R^{1B} is 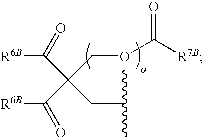
R^{2B} is 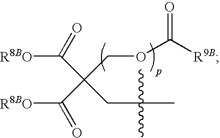
R^{3B} is 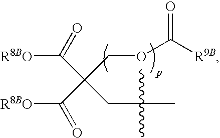
wherein R^{2B} and R^{3B} can be the same or different;
R^{4B} and R^{5B} are independently selected from the group consisting of hydrogen, an optionally substituted C₁₋₄ alkyl, and —C(R^{10B})₂—O—C(═O)R^{11B};
each R^{6B}, each R^{7B}, each R^{8B}, each R^{9B}, each R^{10B} and each R^{11B} are each independently hydrogen or an optionally substituted C₁₋₄-alkyl;
each o is independently 1 or 2; and
each p is independently 1 or 2.

14. The compound of claim 13, wherein each R^{6B} is an optionally substituted C₁₋₄ alkyl.

15. The compound of claim 13, wherein each R^{7B} is an optionally substituted C₁₋₄ alkyl.

16. The compound of claim 13, wherein each R^{8B} is an optionally substituted C₁₋₄ alkyl.

17. The compound of claim 13, wherein each R^{9B} is an optionally substituted C₁₋₄ alkyl.

18. The compound of claim 13, wherein each R^{1B}, R^{2B} and R^{3B} are each independently:

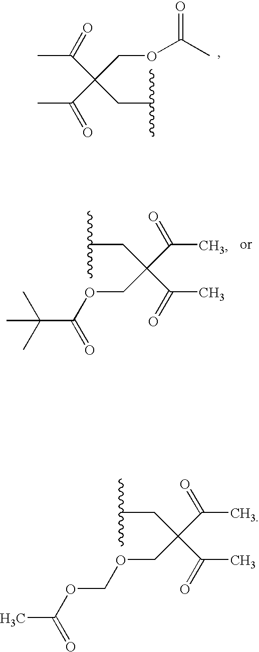

19. A pharmaceutical composition comprising a compound of claim 1, and a pharmaceutically acceptable carrier, diluent, excipient or combination thereof.

20. A method of ameliorating or treating a viral infection comprising administering to a subject suffering with a viral infection a therapeutically effective amount of a compound of claim 1.
